(19) United States
(12) Patent Application Publication
(10) Pub. No.: US 2018/0221393 A1
(43) Pub. Date: Aug. 9, 2018

(54) CXCR4 BINDING AGENTS FOR TREATMENT OF DISEASES

(71) Applicants: Biokine Therapeutics Ltd., Nes Ziona (IL); BioLineRx Ltd., ModiIn (IL)

(72) Inventors: Amnon PELED, Tel-Aviv (IL); Yaron PEREG, Shoham (IL); Michal ABRAHAM, Mevasseret Zion (IL); Shiri KLEIN SILBERMAN, ModiIn (IL)

(21) Appl. No.: 15/749,558

(22) PCT Filed: Aug. 2, 2016
(86) PCT No.: PCT/IL2016/050845

§ 371 Date: Feb. 1, 2018

Related U.S. Application Data
(60) Provisional application No. 62/200,177, filed on Aug. 3, 2015.

(51) Int. Cl. A61K 31/635; A61P 35/00; A61K 45/06; A61K 38/16; A61P 31/18; C12N 15/113

(52) U.S. Cl. CPC A61K 31/635; A61P 35/00; A61K 45/06; C12N 2310/141; A61P 31/18; C12N 15/1132; A61K 38/16

(57) ABSTRACT

A method of treating cancer in a subject is disclosed. The method comprises administering to the subject:
(i) a therapeutically effective amount of an inhibitory agent that down-regulates an amount of a polypeptide selected from the group consisting of BCL-2, MCL-1 and cyclin D1; and
(ii) a therapeutically effective amount of a CXCR4 binding agent that increases the expression of miR-15a and/or 16-1, thereby treating the cancer.

### FIELD AND BACKGROUND OF THE INVENTION

The present invention, in some embodiments thereof, relates CXCR4 binding agents that up-regulate miR-15a and miR-16-1, and uses thereof.

CXCR4 is a G-protein coupled receptor that mediates the activity of CXC chemokines. To date, its only identified cognate ligand is CXCL12, also known as Stromal Cell-Derived Factor-1 (SDF-1). CXCR4 plays an important role in mammalian development, mediating the migration and motility of tissue and hematopoietic stem and progenitor cells. Both CXCR4 and SDF-1 knock-out mice show embryonic lethality with essentially identical phenotypes involving tissue and vascular malformations, supporting the hypothesis that CXCR4 is the key receptor for the activity of SDF-1 (CXCR7 is a second known receptor of SDF-1). CXCR4 continues to be broadly expressed in the adult, with high levels detected on bone marrow stem and progenitor cells, various circulating lymphocytes (B-cells, activated T-cells), as well as endothelial precursor cells, and tissue macrophages and fibroblasts.

CXCR4 is likely to play a pleiotropic role in human cancer. Its expression is upregulated in many tumor types, including cancers of the breast, lung, colon, pancreas, brain, prostate, ovary, as well as hematopoietic cancers. Some literature reports suggest that SDF-1 may act through CXCR4 as a growth and/or survival factor for some tumors. In models of metastatic cancer, CXCR4 positive tumors were shown to metastasize to distant sites, and this activity was inhibited by agents that silence the CXCR4 gene or antibodies that block CXCR4 or SDF-1. Consistent with this view, many common sites of metastasis in human cancer, such as bone marrow, lung, lymph node, and liver, express high levels of SDF-1. CXCR4 is expressed on stem cell-like or tumor initiating subpopulations of many tumors, and may mediate the ability of these cells to support the recurrence and metastatic spread of cancers. Furthermore, CXCR4 is expressed on endothelial precursor cells (EPCs), and its activity is required for incorporation of EPCs into functional vessels during angiogenesis. This may make a significant contribution to the vascularization and survival of tumors. CXCR4 signaling can also lead to induction of pro-angiogenic cytokines (e.g., VEGF), as well as integrins, adhesion molecules and matrix degrading enzymes that may mediate invasion by tumor cells. Lastly, CXCR4 expression is detected on tumor infiltrating lymphocytes and fibroblasts, as well as tumor associated macrophages. These cells tend to suppress immune recognition and attack on the tumor, and remodel the tumor microenvironment to encourage tumor growth and metastasis.

The multiple roles of CXCR4 in tumor growth, vascularization, and metastasis, and its broad expression in many common tumor types, make this receptor an attractive target for therapeutic intervention using inhibitory agents.

4F-benzoyl-TN14003 (also known as BKT140, hereinafter BL-8040), is a 14-residue bio stable synthetic peptide developed as a specific CXCR4 antagonist. It has been shown that BL-8040 binds the CXCR4 receptor with high affinity and long receptor occupancy. BL-8040 was found to be toxic against several tumors such as myeloid leukemia, hematopoietic tumors and non-small cell lung cancer (International Patent Application No. IL2014/050939 and International Patent Application Publication No. WO2013/16089556053 and WO2008/075370).

US Patent Application No. 20070238115 teaches use of CXCL12 for treatment of carcinoma.

US Patent Application No. 20130281423 teaches the use of CXCL12 inhibitors for reducing the resistance of a tumor cell to a chemotherapeutic agent.

Singh et al., Mol Cancer Ther January 2009 8; 178-184 teaches gp120-IIIB for the treatment of prostate cancer.

Danilov, A. V., et al., (2013). Expert Review of Anticancer Therapy, 13(9), 1009-1012 teaches the use of plerixafor in combination with BCl-2 inhibitors for the treatment of chronic lymphocytic leukemia (CLL).

### SUMMARY OF THE INVENTION

According to an aspect of some embodiments of the present invention there is provided a method of treating cancer in a subject comprising administering to the subject:

(i) a therapeutically effective amount of an inhibitory agent that down-regulates an amount of a polypeptide selected from the group consisting of BCL-2, MCL-1 and cyclin D1; and

(ii) a therapeutically effective amount of a CXCR4 binding agent that increases the expression of miR-15a and/or 16-1, thereby treating the cancer.

According to an aspect of some embodiments of the present invention there is provided a method of rendering a BCl-2 antagonist resistant subject more susceptible to treatment with a BCl-2 antagonist comprising administering to the subject a therapeutically effective amount of a CXCR4 binding agent that increases the expression of miR-15a and/or 16-1, thereby treating the cancer.

According to an aspect of some embodiments of the present invention there is provided a method of treating a neurodegenerative disease or a cancer comprising administering to the subject a therapeutically effective amount of CXCL12, thereby treating the cancer, wherein the cancer is not a carcinoma.

According to an aspect of some embodiments of the present invention there is provided a method of identifying an agent useful for the treatment of cancer or a neurodegenerative disease comprising:

(a) contacting a cell population with the agent;

(b) determining whether the agent binds to CXCR4; and

(c) determining whether the agent modulates the level of miRNA-15a and/or miRNA-16-1, wherein an agent which binds to CXCR4 and increases the level of miRNA-15a or 16-1 is indicative that the agent is useful for the treatment of cancer or the neurodegenerative disease.

According to an aspect of some embodiments of the present invention there is provided a method of identifying whether a CXCR4 binding agent is useful for the treatment of cancer or a neurodegenerative disease comprising:

(a) contacting a cell population with the agent; and

(b) determining whether the agent modulates the level of miRNA-15a and/or miRNA-16-1, wherein an agent which increases the level of miRNA-15a or 16-1 is indicative that the agent is useful for the treatment of cancer or the neurodegenerative disease.

According to an aspect of some embodiments of the present invention there is provided a method of treating cancer or a neurodegenerative disease in a subject in need thereof, comprising administering to the subject a therapeutically effective amount of gp120, thereby treating the cancer or neurodegenerative disease, with the proviso that the cancer is not prostate cancer.

According to an aspect of some embodiments of the present invention there is provided a method of treating HIV in a subject comprising administering to the subject a polynucleotide agent which is complementary to a nucleotide sequence of human miR-15a and/or 16-1, thereby treating the HIV in the subject.

According to some embodiments of the invention, the inhibitory agent is co-administered with the CXCR4 binding agent.

According to some embodiments of the invention, the inhibitory agent is administered following administration of the CXCR4 binding agent.

According to some embodiments of the invention, the inhibitory agent is co-formulated with the CXCR4 binding agent.

According to some embodiments of the invention, the inhibitory agent is a nucleic acid agent.

According to some embodiments of the invention, the nucleic acid agent is selected from the group consisting of an antisense RNA, siRNA and a ribozyme.

According to some embodiments of the invention, the inhibitory agent is a peptide agent.

According to some embodiments of the invention, the inhibitory agent is a BCl-2 antagonist.

According to some embodiments of the invention, the BCl-2 antagonist is selected from the group consisting of Oblimersen, SPC-2996, RTA-402, Gossypol, AT-101, Obatoclax mesylate, A-371191, A-385358, A-438744, ABT-737, ABT-199, AT-101, BL-11, BL-193, GX-15-003, 2-Methoxyantimycin A3, HA-14-1, KF-67544, Purpurogallin, TP-TW-37, YC-137 and Z-24.

According to some embodiments of the invention, the BCl-2 antagonist is ABT-199.

According to some embodiments of the invention, the method further comprises administering to the subject an additional chemotherapeutic agent.

According to some embodiments of the invention, the additional chemotherapeutic agent is rituximab and Bendamastin.

According to some embodiments of the invention, the CXCR4 binding agent binds to the CXCL12 binding site of CXCR4.

According to some embodiments of the invention, the agent is BL8040.

According to some embodiments of the invention, the CXCR4 binding agent is gp120 or CXCL-12.

According to some embodiments of the invention, the cancer is selected from the group consisting of a hematological cancer, neuroblastoma, retinoblastoma bladder cancer, esophageal carcinoma, sarcomas, colorectal cancer, melanoma, parathyroid adenoma, and breast cancer.

According to some embodiments of the invention, the hematological cancer is selected from the group consisting of hematological malignancy is selected from the group consisting of Chronic Myelogenous Leukemia (CML), CML accelerated phase, or blast crisis, multiple myeloma, Hypereosinophilic Syndrome (HES), myelodysplastic syndrome (MDS), acute lymphocytic leukemia (ALL), acute myeloid leukemia (AML), acute promyelocytic leukemia (APL), chronic lymphocytic leukemia (CLL), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML with trilineage myelodysplasia (AML/TMDS), mixed lineage leukemia (MLL), myeloproliferative disorders (MPD), multiple myeloma, (MM) and myeloid sarcoma.

According to some embodiments of the invention, the BCl-2 antagonist is selected from the group consisting of Oblimersen, SPC-2996, RTA-402, Gossypol, AT-101, Obatoclax mesylate, A-371191, A-385358, A-438744, ABT-737, ABT-199, AT-101, BL-11, BL-193, GX-15-003, 2-Methoxyantimycin A3, HA-14-1, KF-67544, Purpurogallin, TP-TW-37, YC-137 and Z-24.

According to some embodiments of the invention, the BCl-2 antagonist is ABT-199.

According to some embodiments of the invention, the CXCR4 binding agent binds to the CXCL12 binding site of CXCR4.

According to some embodiments of the invention, the CXCR4 binding agent is BL8040.

According to some embodiments of the invention, the CXCR4 binding agent is gp120 or CXCL-12.

According to some embodiments of the invention, the subject has a cancer selected from the group consisting of a hematological cancer, neuroblastoma, retinoblastoma bladder cancer, esophageal carcinoma, sarcomas, colorectal cancer, melanoma, parathyroid adenoma and breast cancer.

According to some embodiments of the invention, the hematological cancer is selected from the group consisting of hematological malignancy is selected from the group consisting of Chronic Myelogenous Leukemia (CML), CML accelerated phase, or blast crisis, multiple myeloma, Hypereosinophilic Syndrome (HES), myelodysplastic syndrome (MDS), acute lymphocytic leukemia (ALL), acute myeloid leukemia (AML), acute promyelocytic leukemia (APL), chronic lymphocytic leukemia (CLL), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myeloproliferative disorders (MPD), multiple myeloma, (MM) and myeloid sarcoma.

According to some embodiments of the invention, the cancer is a hematological cancer.

According to some embodiments of the invention, the neurodegenerative disease is selected from the group consisting of Alzheimer's disease (AD), Parkinson's disease, and frontotemporal dementias (FTD).

According to some embodiments of the invention, the determining whether the agent modulates the level of miRNA-15a and/or miRNA-16-1 is effected by analyzing expression of a target gene of the miRNA-15a and/or miRNA-16-1.

According to some embodiments of the invention, the target gene is selected from the group consisting of BCL-2, MCL-1 and cyclin D1.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A illustrates that over-expression of CXCR4 increases the level of pERK. Cells (SKNBE, RPMI and PC3 cells) were stained with PE-conjugated anti-human CXCR4 clone 12G5. Expression of CXCR4 was measured on SKNBE, RPMI and PC3 cells (purple) and in cells genetically modified to over-express CXCR4 (green line) by FACS analysis. Over-expression of CXCR4 induced an elevation in the level of pERK in SKNBE, PC3 and RPMI cells as demonstrated by Western Blot. Cells were cultured with 1% FCS RPMI medium for 24 hr. Whole-cell lysates were assayed by Western blots and levels of pERK were detected and normalized to β-actin to ensure equal protein loading.

FIG. 1B illustrates that over-expression of CXCR4 up-regulates the expression of Mir-15a and Mir-16-1. Expression of Mir-15a and Mir 16-1 was assessed by RT-PCR in SKNBE, RPMI and PC3 cells following over-expression of CXCR4.

FIG. 1C illustrates that over-expression of CXCR4 down-regulates the mRNA expression of the Mir-15a and Mir-16-1target genes: BCL-2, MCL-1 and CyclinD1 as analyzed by RT-PCR. In SKNBE cells, over-expression of CXCR4 down-regulated the expression of BCL-2 mRNA, while the mRNA expression of CyclinD1 and MCL-1 was not affected. Over-expression of CXCR4 down-regulated the expression of BCL-2, MCL-1 and CyclinD1 mRNA in PC3 cells. In RPMI cells, over-expression of CXCR4 down-regulated the expression of MCL-1 and CyclinD1 mRNA, but not the expression of BCL-2.

FIG. 1D illustrates that over-expression of CXCR4 down-regulates BCL-2 protein expression in SKNBE cells, as analyzed by Western Blot (WB) and by FACS analysis. Whole-cell lysates were assayed by Western blots and levels of BCL-2 were detected and normalized to the β-actin detection to confirm equal protein loading. Mean fluorescence intensity (MFI) of BCL-2 was evaluated by FACS following intracellular staining of anti-Human BCL-2. BCL-2 protein was down-regulated following over-expression of CXCR4 in PC3 cells but did not change in RPMI-8226 cells as demonstrated by Western Blot.

FIG. 2A illustrates that SDF-1 (CXCL12) induces the expression of miR15a and downregulates the expression of BCL2 mRNA in SKNBE neuroblastoma cells.

SKNBE cells were incubated in the presence of SDF-1 (100 ng/ml) for 24 hr in 10% FCS medium. Expression of Mir-15a and mRNA of BCL-2 were evaluated by RT-PCR.

FIG. 2B illustrates that over-expression of CXCR4 transforms SKNBE neuroblastoma cells to be un-resistant to ABT199 treatment. SKNBE cells and SKNBE-CXCR4 cells were incubated for 24 hr in 1% FCS RPMI medium in the presence of BCL-2 inhibitor -ABT-199 (0.01, 0.1, 1 and 10 uM). % of dead cells and viability of cells was evaluated using FACS analysis following staining with PI.

FIG. 2C illustrates that treatment of SKNBE cells with CXCL12 attenuates the apoptosis induced by treatment with ABT199.

SKNBE cells were pre-incubated for 24 hr in 10% FCS RPMI medium in the presence of SDF-1 (100 ng/ml). Following 24 hr of incubation, the BCL-2 inhibitor -ABT-199 (0.01, 0.1, 1 and 10 uM) was added to the cells and cultured for another 24 hr in 1% FCS medium. % of dead cells was evaluated using FACS analysis following PI staining.

Red bars—The effect of ABT-199 on viability of SKNBE cells.

Blue bars—The effect of ABT-199 on viability of SKNBE cells following pre-incubation with SDF-1.

FIG. 2D illustrates that treatment of SKNBE with the CXCR4 binding agent, BL8040, inhibits ERK signaling, inducing miR-15a expression and reducing BCL-2 expression levels. Analysis of expression of pERK was performed by Western Blot (WB). SKNBE cells were cultured with 1% FCS RPMI medium for 24 hr after which BL-8040 (20 uM) was added to the cells for 30 minutes or 2 hr. Whole-cell lysates were assayed by WB and levels of pERK were detected and normalized to β-actin to confirm equal protein loading. The level of miR-15a and BCL-2 was assessed by RT-PCR in SKNBE cells following a 24 hr incubation in the presence of BL-8040 (20 uM) with 10% FCS medium.

FIG. 2E illustrates that up-regulation of miR-15a by transfection of SKNBE cells with miR-15a or miR-16-1 induced cell death. The direct effect of miR-15a and mir-16-1 on survival and viability of cells was analyzed following transfection with miR-15a and mirR-16-1 to SKNBE cells. 48 hr following transfection, viability of cells was assessed by FACS following staining with PI.

FIG. 3A illustrates that MV-V4-11 cells are dependent on BCL-2 for their survival in vitro and can be induced to undergo apoptosis using ABT-199. MV4-11, RPMI and K562 cells were incubated for 24 hr in 1% FCS RPMI medium in the presence of the BCL-2 inhibitor ABT-199 (0.01, 0.1, 1 and 10 uM). Viability of cells was evaluated using FACS analysis following PI staining.

FIG. 3B illustrates that treatment of MV4-11 cells with CXCL12 attenuated the apoptosis induced by treatment with ABT199. MV4-11 cells were pre-incubated for 24 hr in 10% FCS RPMI medium in the presence of SDF-1 (100 ng/ml). Following 24 hr of incubation BCL-2 inhibitor -ABT-199 (0.01, 0.1, 1 and 10 uM) was added to the cells and cultured for another 24 hr in 1% FCS medium. % of dead cells was evaluated using FACS analysis following PI staining.

Red bars—The effect of ABT-199 on viability of MV4-11 cells.

Blue bars—The effect of ABT-199 on viability of MV4-11 cells following pre-incubation with SDF-1.

FIG. 3C illustrates that the CXCR4 binding agent, BL8040, induces rapid and dose dependent apoptosis of MV4-11 cells, inhibits ERK signaling and induces miR-15a expression and significantly reduces BCL2/MCL-1/and cyclinD1 expression levels. MV4-11 cells were cultured with 1% FCS RPMI medium for 24 hr. 24 hr after incubation, BL-8040 (8 uM) was added to the cells for 30 minutes or 2 hr. Whole-cell lysates were assayed by WB and levels of pERK were detected and normalized to the β-actin to confirm equal protein loading. MV4-11 cells were incubated for 24 hr in the presence of BL-8040 (8 uM) with 1% FCS medium. Level of miR-15a, mRNA of BCL-2 and MCL-1 was assessed by RT-PCR and the level of BCL-2 and cyclinD1 protein was evaluated by WB.

FIG. 3D illustrates that transfection of MV4-11 cells with miR-15a or miR-16-1 induces cell death. Analysis of the direct effect of miR-15a and mir-16-1 on survival and viability of MV4-11 cells was performed following transfection of miR-15a and miR-16-1 to the cells. 48 hr following transfection, viability of the cells was assessed by FACS following staining with PI. Level of miR-15a and miR-16-1-1 was measured by RT-PCR 24 hr post transfection.

FIG. 3E illustrates that BL8040 was unable to induce cell death in CLL cells that do not express miR-15a116-1. Level of miR-15a was evaluated in cells of CLL patients using RT-PCR. The ability of BL-8040 to kill cells from CLL patients was measured following incubation of cells for 24 hr at 1% FCS medium in the presence of BL-8040 (0.1, 1, 10 and 20 uM). Viability of cells was assessed by FACS following staining with PI.

FIG. 3F illustrates that Gp120 up-regulates miR-15a/16-1 and down regulates BCL2 in CD4+ cells and induces cell death. Gp120 was found to induce dramatic apoptosis of MV4-11 cells. CD4+ T cells were incubated for 24 hr in 1% FCS medium in the presence of SDF-1 (100 ng/ml), BL-8040 (8 uM) or GP120 (1 ug/ml). Level of BCL-2 mRNA was evaluated following 24 hr of incubation by RT-PCR. % of dead cells was assessed by FACS following staining with PI. The effect of Gp120 on viability of MV4-11 cells was measured following incubation with Gp120 (0.1, 1, 10 and 20 ug/ml) for 24 hr at 1% FCS medium. % of dead cells and number of viable cells were assessed by FACS following staining with PI.

FIG. 4A illustrates that in-vivo treatment of BL8040 significantly increases miR-15a and down regulated BCL2 in a neuroblastoma mice model. NSG mice were injected with SKNBE cells into the left adrenal. One week later BL-8040 was SC injected daily for 14 days at a concentration of 400 μg/mouse. 24 hr after the last BL-8040 injection, animals were sacrificed; tumors were harvested, measured and weighed. RNA and total protein were extracted from the tumor and the level of miR-15a was evaluated by qRT-PCR. The level of BCL-2 protein was evaluated by Western blot.

FIG. 4B illustrates that in the in-vivo AML model of MV4-11 cells, reduction in AML cells in the spleen is associated with a significant increase in miR-15a/16-1 and down regulation of BCL2 in human cells. NOD SCID gamma (NSG) mice were engrafted with MV4-11 cells. Engraftment of AML cells was allowed for 3 weeks following transplantation. At day 21, BL-8040 was SC injected into mice at 400 μg/mouse once daily for one or two days (BL8040×1, BL8040×2, respectively). 24 hr after the last injection, the mice were sacrificed and the level of human CD45+ AML cells in the spleen was evaluated by FACS. RNA was extracted from the spleen and the level of miR-15a, Mir-16-1 was evaluated by qRT-PCR. The level of BCL-2 protein was evaluated by Western blot.

### DESCRIPTION OF SPECIFIC EMBODIMENTS OF THE INVENTION

The present invention, in some embodiments thereof, relates to CXCR4 binding agents that up-regulate miR-15a and miR-16-1, and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

CXCR4 is over-expressed in the majority of tumor cells and its expression typically correlates with bad prognosis. The present inventors have now found that over-expression of CXCR4 on tumor cells and simulation of cells with CXCL12 leads to up-regulation of miR-15a/16-1 and consequently down regulation of their target genes BCL-2, MCL-1, and cyclin D1. Furthermore, overexpression of CXCR4 in these cells increases tumorogenesis and shift their oncogenic dependency from BCL2 to CXCR4. The present inventors further showed that that CXCR4 binding agent BL8040 has a similar effect on miR-15a/16-1. Thus, BL8040 induced apoptosis of tumor cell by inhibiting survival signals while keeping apoptotic gene expression low both in vitro and in vivo. The present results suggest that overexpression of CXCR4 may override the survival dependency of tumor cells on BCL-2, MCL-1, and cyclin D1 leading to resistance of tumor cells to inhibition of these pathways.

Thus, according to a first aspect of the present invention there is provided a method of treating cancer in a subject comprising administering to the subject:

(i) a therapeutically effective amount of an inhibitory agent that down-regulates an amount of a polypeptide selected from the group consisting of BCL-2, MCL-1 and cyclin D1; and

(ii) a therapeutically effective amount of a CXCR4 binding agent that increases the expression of miR-15a and/or 16-1, thereby treating the cancer.

As used herein, the term cancer refers to proliferative diseases including but not limited to carcinoma, lymphoma, blastoma, sarcoma, and leukemia. The cancer may for example be a solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chondrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a particular embodiment, the cancer is neuroblastoma, retinoblastoma bladder cancer, esophageal carcinoma, sarcomas, colorectal cancer, melanoma, parathyroid adenoma, and breast cancer.

According to another embodiment, the cancer is a solid tumor.

According to yet another embodiment, the cancer is hematological malignancy.

The term “hematological malignancy” herein includes a lymphoma, leukemia, myeloma or a lymphoid malignancy, as well as a cancer of the spleen and the lymph nodes. Exemplary lymphomas that are amenable to treatment with the disclosed agents include both B cell lymphomas and T cell lymphomas. B-cell lymphomas include both Hodgkin's lymphomas and most non-Hodgkins lymphomas. Non-limiting examples of B cell lymphomas include diffuse large B-cell lymphoma (DLBCL), follicular lymphoma (FL), mucosa-associated lymphatic tissue lymphoma (MALT), small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, acute myelogenous leukemia (AML; also called acute lymphoid leukemia), chronic myelogenous leukemia (CML), B-cell prolymphocytic leukemia (B-PLL), acute lymphoblastic leukemia (ALL) and myelodysplasia (MDS). Hematological malignancies further include myelomas, such as, but not limited to, multiple myeloma (MM), smoldering multiple myeloma (SMM) and B-cell chronic lymphocytic leukemia (CLL).

According to a particular embodiment, the hematological malignancy is chronic myelogenous leukemia (CML). The term CML includes imatinib-resistant CML, CML tolerant to second/third generation Bcr-Abl TKIs (e.g., dasatinib and nilotinib), imatinib-intolerant CML, accelerated CML, and lymphoid blast phase CML.

Other hematological and/or B cell- or T-cell-associated cancers are encompassed by the term hematological malignancy. For example, hematological malignancies also include cancers of additional hematopoietic cells, including dendritic cells, platelets, erythrocytes, natural killer cells, and polymorphonuclear leukocytes, e.g., basophils, eosinophils, neutrophils and monocytes. It should be clear to those of skill in the art that these pre-malignancies and malignancies will often have different names due to changing systems of classification, and that patients having lymphomas classified under different names may also benefit from the therapeutic regimens of the present invention.

Subjects treated according to this aspect of the present invention are typically mammalian subjects, e.g., humans.

The term “Bcl-2” as used herein refers to the Bcl-2 protein (Swiss Prot ID No. P10415), a member of the Bcl-2 family of proteins (Cory, S., and Adams, J. M., Nature Reviews Cancer 2 (2002) 647-656; Adams, Genes and Development 17 (2003) 2481-2495; Danial, N. N., and Korsmeyer, S. J., Cell 116 (2004) 205-219; Petros, A. M., Biochim Biophys Acta 1644 (2004) 83-94).

The term “Mcl-1” as used herein refers to myeloid cell leukemia sequence 1 (BCL2-related) gene (official symbol: MCL1) or protein (Swiss Prot ID No. Q07820). The protein encoded by the Mcl-1 gene belongs to the Bcl-2 family. Two transcript variants encoding distinct isoforms have been identified. The longer gene product (isoform 1) enhances cell survival by inhibiting apoptosis while the alternatively spliced shorter gene product (isoform 2) promotes apoptosis and is death-inducing.

The term “Cyclin D1” refers to the protein also known as Parathyroid Adenomatosis 1 (Swiss Prot ID No. P24385). Refseq mRNAs include NM_053056.2 and XM_006718653.2.

Following is a list of agents capable of downregulating expression level and/or activity of BCl-2, MCL1 or Cyclin D1. It will be appreciated that the present invention contemplate administering agents that down-regulate expression of BCl-2 alone, MCL1 alone or Cyclin D1 alone, as well as combinations of agents that down regulate BCl-2 and MCL1, BCl-2 and Cyclin D1 or MCL1 and Cyclin D1. In addition, the present invention contemplates administering agents which down-regulate BCl-2, MCL1 and Cyclin D1.

One example, of an agent capable of downregulating BCl-2, MCL1 or Cyclin D1 is an antibody or antibody fragment capable of specifically binding thereto. Preferably, the antibody specifically binds at least one epitope of BCl-2, MCL1 or Cyclin D1. As used herein, the term “epitope” refers to any antigenic determinant on an antigen to which the paratope of an antibody binds.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Downregulation of BCl-2, MCL1 or Cyclin D1 can be also achieved using nucleic acid agents, for example by RNA silencing.

As used herein, the phrase “RNA silencing” refers to a group of regulatory mechanisms [e.g., RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated.

Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., BCl-2, MCL1 or Cyclin D1) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g., embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11.

Some of the nucleotides in the loop can be involved in base-pair interactions with other nucleotides in the loop. Examples of oligonucleotide sequences that can be used to form the loop include 5′-UUCAAGAGA-3′ (SEQ ID NO: 1); Brummelkamp, T. R. et al. (2002) Science 296: 550) and 5′-UUUGUGUAG-3′ (SEQ ID NO: 2); Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Examples of suitable siRNA capable of downregulating BCl-2 include: GCATGCGGCCTCTGTTTGATT (SEQ ID NO: 7).

Examples of suitable siRNA capable of downregulating MCL1 include: TCCAAGGCATGCTTCGGAA (SEQ ID NO: 8).

Examples of suitable siRNA capable of downregulating Cyclin D1 include: CCAGAGUGAUCAAGUGUGATT (SEQ ID NO: 9).

Synthesis of RNA silencing agents suitable for use with some embodiments of the invention can be effected as follows. First, the selected mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

According to another embodiment the RNA silencing agent may be a miRNA or miRNA mimic.

miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target). The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to a miRNA, rather than triggering RNA degradation.

An exemplary miRNA capable of downregulating BCl-2, MCL1 and Cyclin D1 is miR-15 a (SEQ ID NO: 3) UAGCAGCACAUAAUGGUUUGUG, or miR-16-1 (SEQ ID NO: 4) UAGCAGCACGUAAAUAUUGGCG of these miRNAs.

It will be appreciated from the description provided herein above that contacting cells with a miRNA may be effected by transfecting the cells with e.g., the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP.

Another agent capable of downregulating BCl-2, MCL1 or Cyclin D1 is a DNAzyme molecule capable of specifically cleaving an mRNA transcript or DNA sequence of the BCl-2, MCL1 or Cyclin D1.

DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www(dot)asgt(dot)org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of BCl-2, MCL1 or Cyclin D1 can also be effected by using an antisense polynucleotide capable of specifically hybridizing with an mRNA transcript encoding BCl-2, MCL1 or Cyclin D1.

Examples of anti-Bcl-2 antisense nucleotides include Oblimersen and SPC-2996.

Oblimersen is an antisense oligonucleotide that inhibits Bcl-2 expression. The antisense oligonucleotide, its sequence and its preparation are described e.g., in WO 95/08350, WO 1999/051259, WO 2002/017852, WO 2004/056971 and U.S. Pat. No. 5,734,033. Oblimersen (or other synonyms: Genansense, G-3139, Oblimersen sodium) as used herein means Heptadecasodium salt of 18-mer antisense phosphorothioate oligodeoxynucleotide whose sequence is: 5′-TCTCCCAGCGTGCGCCAT-3 (SEQ ID NO: 5); Heptadecasodium salt of antisense oligonucleotide from fragment 32-49nt (start codon region) of the human BCL2 cDNA; P-Thiothymidylyl-(3′-5)-2′-deoxy-P-thiocytidylyl-(3′-5)-P-thiothymidylyl-(3′-5)-2′-deoxy-P-thiocytidylyl-(3′-5′)-2′-deoxy-P-thiocytidylyl-(3′-5)-2′-deoxy-P-thiocytidylyl-(3′-5′)-2′-deoxy-P-thioadenylyl-(3′-5)-2′-deoxy-P-t-hioguanylyl-(3′-5)-2′-deoxy-P-thiocytidylyl-(3 ‘-5)-2’-deoxy-P-thioguanylyl-(3′-5)-P-thiothymidylyl-(3′-5)-2′-deoxy-P-thioguanylyl-(3′-5′)-2′-deoxy-P-thiocytidylyl-(3′-5′)-2′-deoxy-P-thioguanylyl-(3′-5′)-2′-deoxy-P-thiocytidylyl-(3′-5)-2′-deoxy-P-thiocytidylyl-(3′-5)-2′-deoxy-P-thioadenylyl-(3′-5-)-thymidine heptadecasodium salt.

SPC-2996, an antisense oligonucleotide, is a 16-mer antisense phosphorothioate oligonucleotides whose sequence is 5′-CTCCCAACGTGCGCCA-3′ (SEQ ID NO: 6) and in which nucleotides 1, 2, 14 and 15 are locked nucleic acid (LNA) nucleotides with enhanced resistance to nuclease digestion. This antisense LNA oligonucleotide targets nucleotides 33-48 (coding sequence) of human Bcl-2.

Another agent capable of downregulating BCl-2, MCL1 or Cyclin D1 is a ribozyme molecule capable of specifically cleaving an mRNA transcript encoding BCl-2, MCL1 or Cyclin D1. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of regulating the expression of BCl-2, MCL1 or Cyclin D1 genes in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the BCl-2, MCL1 or Cyclin D1 regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Publication Nos. 2003/017068 and 2003/0096980 to Froehler et al, and 2002/0128218 and 2002/0123476 to Emanuele et al, and U.S. Pat. No. 5,721,138 to Lawn.

It will be appreciated that any of the nucleic acid agents described in the present application can be modified to increase efficacy.

The disclosed nucleic acid agents may be composed of naturally occurring nucleobases, sugars, and covalent internucleoside (backbone) linkages as well as having non-naturally-occurring portions that similarly. Such modified or substituted oligonucleotides are often preferred over native forms because of desirable properties such as, for example, enhanced cellular uptake, enhanced affinity for nucleic acid target, and/or increased stability in the presence of nucleases.

The nucleic acid agents disclosed herein can include unmodified RNA and DNA as well as RNA and DNA that have been modified, e.g., to improve efficacy, and polymers of nucleoside surrogates. “Unmodified” nucleic acids refers to a molecule in which the components of the nucleic acid, namely sugars, bases, and phosphate moieties, are the same or essentially the same as that which occur in nature, preferably as occur naturally in the human body. “Modified” nucleic acid, as used herein, refers to a molecule where one or more of the components of the nucleic acid, namely sugars, bases, and phosphate moieties, are different from that which occur in nature, preferably different from that which occurs in the human body.

Nucleic acid agents designed according to the teachings of some embodiments of the invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g., cyanoethyl phosphoramidite followed by deprotection, desalting and purification by for example, an automated trityl-on method or HPLC.

The nucleic acid agents of some embodiments of the invention is of at least 17, at least 18, at least 19, at least 20, at least 22, at least 25, at least 30 or at least 40, bases specifically hybridizable with sequences described hereinabove.

The nucleic acid agents of some embodiments of the invention may comprise heterocyclic nucleosides consisting of purines and the pyrimidines bases, bonded in a 3′ to 5′ phosphodiester linkage.

Preferably used nucleic acid agents are those modified in either backbone, internucleoside linkages or bases, as is broadly described hereinunder.

Specific examples of preferred nucleic acid agents useful according to some embodiments of the invention include nucleic acid agents containing modified backbones or non-natural internucleoside linkages. Nucleic acid agents having modified backbones include those that retain a phosphorus atom in the backbone, as disclosed in U.S. Pat. Nos.: 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified nucleic acid agent backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other nucleic acid agents which can be used according to some embodiments of the invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in some embodiments of the invention are disclosed in U.S. Pat. No: 6,303,374.

Nucleic acid agents of some embodiments of the invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No.: 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications. Still further base substitutions include the non-standard bases disclosed in U.S. Pat Nos. 8,586,303, 8,614,072, 8,871,469 and 9,062,336, all to Benner et al: for example, the non-standard dZ:dP nucleobase pair which Benner et al has shown can be incorporated into DNA by DNA polymerases to yield amplicons with multiple non-standard nucleotides.

Another agent capable of downregulating BCl-2, MCL1 or Cyclin D1 or would be any molecule which binds to and/or cleaves. Such molecules can be antagonists, or inhibitors.

Examples of agents capable of downregulating MCL1 are described n US Patent Application No. 20120172285, the contents of which are incorporated herein by reference.

Examples of cyclin D1 inhibitors include, but are not limited to monoterpenes, nordihydroguaiaretic acid, acyclic retinoid (ACR), sesquicillin, sulinac (an NSAID), methylglyoxal bis(cyclopentylamidinohydrazone), ANXA-1, FR-901228 (a cyclic peptide inhibitor of histone deacetylase), simvastatin (mevalonate/protein prenylation inhibitor), cerivastatin (inhibitor of HMG-CoA reductase), (−)-enantiomer of glossypol (polyphenolic pigment present in cottonseed), ursolic acid (pentacyclictriterpenoid), 14-epi-analogues of 1,25-dihydroxyvitamin D3, tangeritin(5,6,7,8,4′-pentamethoxyflavone), purvalanol A (protein kinase inhibitor), tetrandrine, deoxybouvardin, lycopene, podophyllotoxin GL331, resveratrol, silymarin, epigallocatechin-3-gallate (EGCG), piceatannol, exisulind, oxamflatin, androstanes and androstenes and prostaglandin A2.

The term “Bcl-2 inhibitors” as used herein refers to agents which inhibit the Bcl-2 protein interaction activity either by the inhibition of the phosphorylation of Bcl-2 (“Bcl-2 protein phosphorylation inhibitors”) such as e.g., RTA-402 or by binding to the Bcl-2 protein and thus disruption of the Bad/Bcl-2 complex (these are referred to as “Bcl-2 protein binding inhibitors”). Preferably said Bcl-2 inhibitors are Bcl-2 protein binding inhibitors. The Bcl-2 inhibitory activity via direct binding of such Bcl-2 protein binding inhibitors can be measured via a competitive binding assay. Thus the IC50 of the inhibition of the Bcl-2 protein activity can be determined in an homogenous time resolved fluorescence (HTRF) Assay. Preferably the IC50 of anti-Bcl-2 inhibitory activity is 5 μM or less, more preferably 1 μM or less. Such Bcl-2 protein binding inhibitors include compounds such as Gossypol, AT-101, ABT-199 (also known as Venetoclax), Obatoclax mesylate, A-371191, A-385358, A-438744, ABT-737, ABT-263, AT-101, BL-11, BL-193, GX-15-003, 2-Methoxyantimycin A3, HA-14-1, KF-67544, Purpurogallin, TP-TW-37, YC-137 and Z-24.

“RTA-402” as used herein means CDDO-Me, the methyl ester of the C28-triterpenoid: oleanane triterpenoid 2-cyano-3,12-dioxoolean-1,9-dien-28-oic acid (CDDO) (See e.g., Honda, T., Rounds B V Bore, L., et al. J Med Chem. 43 (2000) 4233-4246), which blocks Bcl-2 protein phosphorylation (Konopleva, M., et al., Blood 99 (2002) 326-35).

Venetoclax (CAS Reg. No. 1257044-40-8; ABT-199; GDC-0199; RG-7601, AbbVie Inc., Genentech Inc.) is a BH3-mimetic drug designed to block the function of the protein Bcl 2 and is in Phase 3 clinical trials for the treatment of Multiple myeloma, Chronic lymphocytic leukemia, Systemic lupus erythematosus, Diffuse large B-cell lymphoma, Acute myelogenous leukemia, and Non-Hodgkin lymphoma (Souers et al Nat Med. 2013 Jan. 6. doi: 10.1038/nm.3048). Venetoclax has the IUPAC name: 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfony-1)-2-(1H-pyrrolo[2,3-b]pyridin-5-yloxy)benzamide.

ABT-737 as used herein means N-[4-[4-(4′-Chlorobiphenyl-2-ylmethyl)piperazin-1-yl]benzoyl]-3-[3-(dimethylamino)-1(R)-(phenylsulfanylmethyl)propylamino]-4-nitrobenzenesulfonamide; 4-[4-(4′-Chlorobiphenyl-2-ylmethyl)piperazin-1-yl]-N-[3-[3-(dimethylamino)-1(R)-(phenylsulfanylmethyl)propylamino]-4-nitrophenylsulfonyl]benzamide, a Bcl-2 inhibitor of formula I, which is described in WO 2006/099667 or Corey, S., et al., Cancer Cell 8 (2005) 5-6.

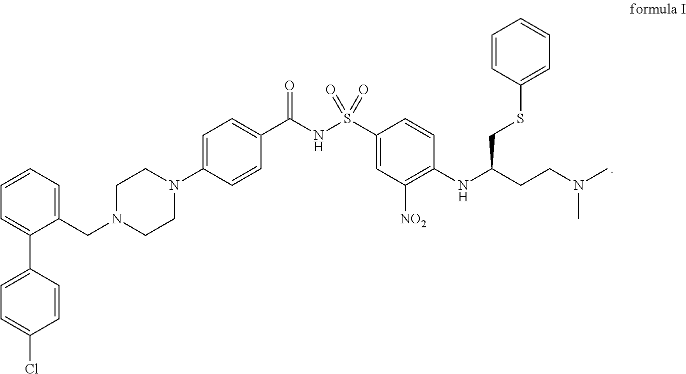 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

ABT-263 as used herein means a Bcl-2 inhibitor of formula II, which is described in US 2007/027,135,

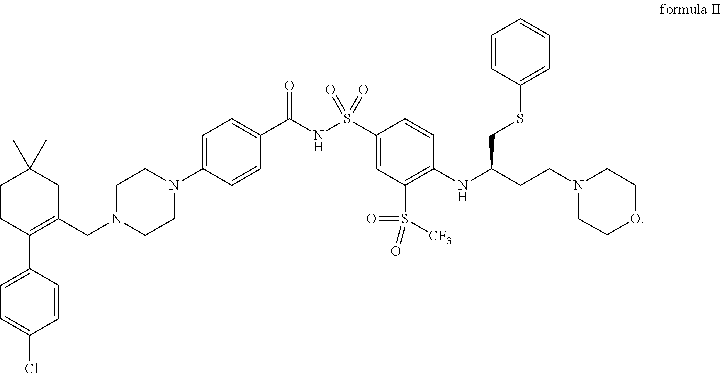 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

A-371191 as used herein means a Bcl-2 inhibitor of formula III,

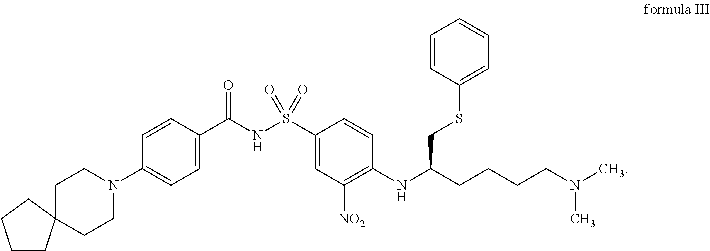 [SMILES: CN(C)CCCC[C@H](CSc1ccccc1)Nc1ccc(S(=O)(=O)NC(=O)c2ccc(N3CCC4(CCCC4)CC3)cc2)cc1[N+](=O)[O-]]

A-385358 as used herein means [(R)-4-(3-dimethylamino-1-phenylsulfanylmethyl-propylamino)-N-[4-(4,4-dimethyl-piperidin-1-yl)-benzoyl]-3-nitrobenzene-sulfonamide (as e.g., disclosed in Shoemaker, A. R., et al., Cancer Research 66 (2006) 8731-8739) a Bcl-2 inhibitor of formula IV,

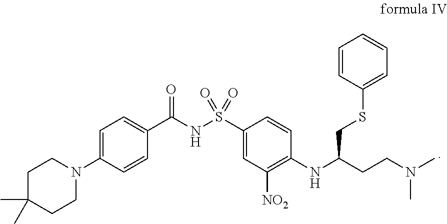

Gossypol as used herein means either a racemic mixture of (+)-Gossypol or (−)-Gossypol (a Bcl-2 inhibitor of formula V), or pure (+)-Gossypol or (−)-Gossypol, preferably Gossypol refers to pure (−)-Gossypol.

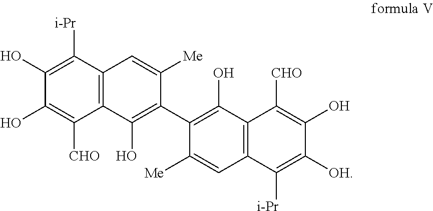 [SMILES: Cc1cc2c(C(C)C)c(O)c(O)c(C=O)c2c(O)c1-c1c(C)cc2c(C(C)C)c(O)c(O)c(C=O)c2c1O]

AT-101 as used herein means clinical lead compound of Ascenta Therapeutics AT-101, a Bcl-2 inhibitor and derivative of R(−)-gossypol.

Obatoclax mesylate (or other synonyms: GX-015-070; or GX15-070) as used herein means 2-[2-(3,5-Dimethyl-1H-pyrrol-2-ylmethylene)-3-methoxy-2H-pyrrol-5-yl]-1H-indole methanesulfonate, a Bcl-2 inhibitor, which is described e.g., in WO 2004/106328, WO 2006/089397 and Walensky, L. D., Cell Death and Differentiation, 13 (2006) 1339-1350. TW-37 as used herein means a Bcl-2 inhibitor of formula VI,

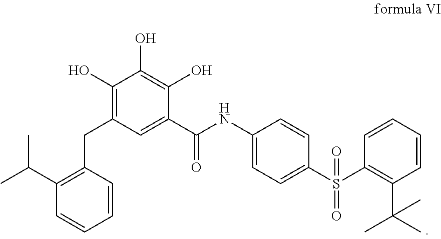 [SMILES: CC(C)c1ccccc1Cc1cc(C(=O)Nc2ccc(S(=O)(=O)c3ccccc3C(C)(C)C)cc2)c(O)c(O)c1O]

BL-193 as used herein means a Bcl-2 inhibitor of formula VII,

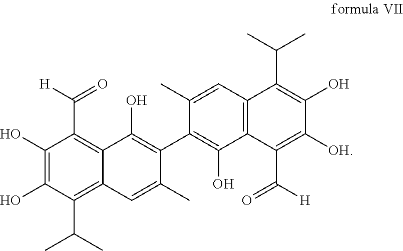 [SMILES: Cc1cc2c(C(C)C)c(O)c(O)c(C=O)c2c(O)c1-c1c(C)cc2c(C(C)C)c(O)c(O)c(C=O)c2c1O]

NSC-719664 as used herein means 2-Methoxy-Antimycin A3, a Bcl-2 inhibitor derived from Antimycin A₃.

YC-137 is described e.g., in Walensky, L. D., Cell Death and Differentiation 13 (2006) 1339-1350.

Purpurogallin is described e.g., in Walensky, L. D., Cell Death and Differentiation 13 (2006) 1339-1350.

HA-14-1 is described e.g., in Walensky, L. D., Cell Death and Differentiation 13 (2006) 1339-1350.

Z-24 as used herein means 3Z-3-[(1H-pyrrol-2-yl)-methylidene]-1-(1-piperidinylmethyl)-1,3-2H-indol-2-one, a Bcl-2 inhibitor of formula VIII,

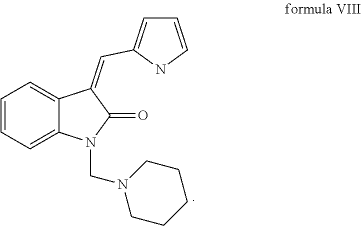 [SMILES: O=C1/C(=C\c2ccc[nH]2)c2ccccc2N1CN1CCCCC1]

As mentioned, the method of this aspect of the present invention also comprises administering to the subject a therapeutically effective amount of a CXCR4 binding agent that increases the expression of miR-15a and/or miR16-1, thereby treating the cancer.

Methods of analyzing the expression level of miR-15a or miR-16-1 are known in the art and include direct analysis of the expression level of these miRNAs or analyzing expression of their target genes, including for example Bcl-2, cyclin D1 and Mcl1, as described herein above.

Exemplary methods for detection of the level of the miRNA (miRNA-15a or 16-1) can be effected using various methods known in the art, including RNA-based hybridization methods (e.g., Northern blot hybridization, RNA in situ hybridization and chip hybridization) and reverse transcription-based detection methods (e.g., RT-PCR, quantitative RT-PCR, semi-quantitative RT-PCR, real-time RT-PCR, in situ RT-PCR, primer extension, mass spectroscopy, sequencing, sequencing by hybridization, LCR (LAR), Self-Sustained Synthetic Reaction (3SR/NASBA), Q-Beta (Qb) Replicase reaction, cycling probe reaction (CPR), a branched DNA analysis, and detection of at least one nucleic acid change).

Total cellular RNA can be isolated from a biological sample using any suitable technique such as the single-step guanidinium-thiocyanate-phenol-chloroform method described in Chomczynski and Sacchi, Anal. Biochem. 162:156-159 (1987) or by using kits such as the Tri-Reagent kit (Sigma).

Following is a non-limiting list of RNA-based hybridization methods which can be used to detect the miRNA of the present invention.

Northern Blot analysis—This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA, RNA or oligonucleotide (composed of deoxyribo or ribonucleotides) probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RNA in situ hybridization stain—In this method DNA, RNA or oligonucleotide (composed of deoxyribo or ribonucleotides) probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

Hybridization to oligonucleotide arrays: The nucleic acid sample which includes the candidate region to be analyzed is isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Probes that perfectly match a sequence of the nucleic acid sample generally produce stronger signals than those that have mismatches. Since the sequence and position of each probe immobilized on the chip is known, the identity of the nucleic acid hybridized to a given probe can be determined.

Preferably, the oligonucleotide probes utilized by the various hybridization techniques described hereinabove are capable of hybridizing to miRNA 15 or 16 under stringent hybridization conditions.

By way of example, hybridization of short nucleic acids (below 200 by in length, e.g., 17-40 by in length) can be effected by the following hybridization protocols depending on the desired stringency; (i) hybridization solution of 6.times.SSC and 1% SDS or 3 M TMAC1, 0.01 M sodium phosphate (pH 6.8), 1 mM EDTA (pH 7.6), 0.5% SDS, 100 .mu·g/ml denatured salmon sperm DNA and 0.1% nonfat dried milk, hybridization temperature of 1-1.5.degree. C. below the Tm, final wash solution of 3 M TMAC1, 0.01 M sodium phosphate (pH 6.8), 1 mM EDTA (pH 7.6), 0.5% SDS at 1-1.5.degree. C. below the Tm (stringent hybridization conditions) (ii) hybridization solution of 6.times.SSC and 0.1% SDS or 3 M TMAC1, 0.01 M sodium phosphate (pH 6.8), 1 mM EDTA (pH 7.6), 0.5% SDS, 100 .mu·g/ml denatured salmon sperm DNA and 0.1% nonfat dried milk, hybridization temperature of 2-2.5.degree. C. below the Tm, final wash solution of 3 M TMAC1, 0.01 M sodium phosphate (pH 6.8), 1 mM EDTA (pH 7.6), 0.5% SDS at 1-1.5.degree. C. below the Tm, final wash solution of 6.times.SSC, and final wash at 22.degree. C. (stringent to moderate hybridization conditions); and (iii) hybridization solution of 6.times.SSC and 1% SDS or 3 M TMAC1, 0.01 M sodium phosphate (pH 6.8), 1 mM EDTA (pH 7.6), 0.5% SDS, 100 .mu·g/ml denatured salmon sperm DNA and 0.1% nonfat dried milk, hybridization temperature at 2.5-3.degree. C. below the Tm and final wash solution of 6.times.SSC at 22.degree. C. (moderate hybridization solution).

For example, a micro-RNA molecule having a nucleic acid sequence as set forth in SEQ ID NO:3 (miR-15a) can be detected using an oligonucleotide probe having a nucleic acid sequence as set forth in SEQ ID NO:10 (uagcagcacauaaugguuugug). For example, a micro-RNA molecule having a nucleic acid sequence as set forth in SEQ ID NO:4 (miR-16-1) can be detected using an oligonucleotide probe having a nucleic acid sequence as set forth in SEQ ID NO:11 (uagcagcacguaaauauuggcg).

MiR-15a and 16-1 can be also detected using a reverse-transcription based method. Reverse-transcription utilizes RNA template, primers (specific or random), reverse transcriptase (e.g., MMLV-RT) and deoxyribonucleotides to form (i.e., synthesize) a complementary DNA (cDNA) molecule based on the RNA template sequence. Once synthesized, the single strand cDNA molecule or the double strand cDNA molecule (which is synthesized based on the single strand cDNA) can be used in various DNA based detection methods such as RT-PCR analysis.

According to one embodiment, the expression of miR-15a and/or 16-1 is increased by at least 10%, 20% 30%, 40%, 50%, 60%, 70, %, 80%, 90% or more than the level of these miRNAs in the absence of the CXCR4 binding agent.

According to another embodiment, the expression of example Bcl-2, cyclin D1 and Mcl1 is decreased by at least 10%, 20% 30%, 40%, 50%, 60%, 70, %, 80%, 90% or more than the level of these genes in the absence of the CXCR4 binding agent.

According to a particular embodiment, the CXCR4 binding agent binds to the CXCL12 binding site of CXCR4.

Thus, in one embodiment the CXCR4 binding agent is SDF-1α (UniProt No. UniProtKB: P48061).

As used herein the term “SDF-1α” (stromal cell-derived factor-1 alpha) refers to at least an active portion of a mammalian (e.g., human) C-X-C chemokine polypeptide (also designated CXCL12) having at least one functional property of SDF-1α (e.g., binding to CXCR4). Examples of SDF-1α amino acid sequences are set forth in SEQ ID NOs: 12 or 13 and in GenBank Accession Nos. NP_000600, NP_001029058, NP_954637 (encoded by GenBank Accession Nos. NM_000609 and NM_199168).

Preferably, the SDF-1 of this aspect of the present invention binds to CXCR4 and has an amino acid sequence at least 90% homologous, 91% homologous, 92% homologous, 93% homologous, 94% homologous, 95% homologous, 96% homologous, 97% homologous, 98% homologous, 99% homologous to the sequence as set forth in SEQ ID NO: 12.

Any SDF-1α known in the art can be used in accordance with the teachings of the present invention. For example, recombinant human SDF-1α (CXCL12) is available from ProSpec-Tany TechnoGene Ltd, Catalog No. CHM-262; recombinant human SDF-1α from Cell Sciences, Catalog Nos. CRS000A, CRS000B and CRS000C; and recombinant human SDF-1α, 125I Conjugated/Tagged from PerkinElmer, Catalog Nos. NEX346025UC and NEX346005UC.

According to another embodiment, the CXCR4 binding agent is gp120.

The term “gp120” refers to the full length immunodeficiency virus glycoprotein that is typically about 120 kDa in size and corresponding to the 5′ half of the viral Env protein, or fragments (i.e. peptides) thereof that contain binding sites for CXCR4.

According to still another embodiment, the CXCR4 binding agent is an antibody that binds to CXCR4. According to this embodiment, the antibody is a monoclonal antibody that recognizes human CXCR4. Preferably, the antibody is humanized.

According to still another embodiment, the CXCR4 binding agent is 4F-benzoyl-TN14003 (SEQ ID NO: 14), also known as BKT140, hereinafter BL-8040), or an analog thereof, belonging to the T-140 peptide family.

Peptide analogs are structurally and functionally related to the peptides disclosed in patent applications WO 2002/020561, WO 2004/020462 and U.S. Provisional Application 62/164,076, also known as “T-140 analogs”.

The inhibitory agent (as described herein above) can be administered immediately prior to (or after) the CXCR4 binding agent, on the same day as, one day before (or after), one week before (or after), one month before (or after), or two months before (or after) the described compounds, and the like.

The inhibitory agents (as described herein above) and the CXCR4 binding agents can be administered concomitantly, that is, where the administering for each of these reagents can occur at time intervals that partially or fully overlap each other. The inhibitory agents described herein and the CXCR4 binding agents can be administered during time intervals that do not overlap each other. For example, the inhibitory agent can be administered within the time frame of t=0 to 1 hours, while the CXCR4 binding agent can be administered within the time frame of t=1 to 2 hours. Also, the inhibitory agent can be administered within the time frame of t=0 to 1 hours, while the CXCR4 binding agents can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the CXCR4 binding agents can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours.

The CXCR4 binding agents and the inhibitory agents are typically provided in combined amounts to achieve therapeutic effectiveness. This amount will evidently depend upon the particular compound selected for use, the nature and number of the other treatment modality, the condition(s) to be treated, prevented and/or palliated, the species, age, sex, weight, health and prognosis of the subject, the mode of administration, effectiveness of targeting, residence time, mode of clearance, type and severity of side effects of the pharmaceutical composition and upon many other factors which will be evident to those of skill in the art.

In one exemplary embodiment, the amount of CXCR4 binding agent is used in an amount below the minimum dose required for therapeutic effectiveness when used as a single therapy (e.g., 10-99%, preferably 25 to 75% of that minimum dose). This allows for reduction of the side effects caused by the CXCR4 binding agent but the therapy is rendered effective because in combination with the inhibitory agents disclosed herein, the combinations are effective overall.

In another exemplary embodiment, the amount of the inhibitory agent is used in an amount below the minimum dose required for therapeutic effectiveness when used as a single therapy (e.g., 10-99%, preferably 25 to 75% of that minimum dose). This allows for reduction of the side effects caused by the inhibitory agent but the therapy is rendered effective because in combination with the CXCR4 binding agents disclosed herein, the combinations are effective overall.

In one embodiment, the inhibitory agents and the CXCR4 binding agents are synergistic with respect to their dosages. That is to say that the effect provided by the combination is greater than would be anticipated from the additive effects of the two agents when used separately. In an alternative but equally preferred embodiment, the inhibitory agents and the CXCR4 binding agents are synergistic with respect to their side effects. That is to say that the side-effects caused by the combination of the two agents are less than would be anticipated when the equivalent therapeutic effect is provided by either the agents when used separately.

It will be appreciated that the two agents may be administered together with additional anti-cancer agents.

Exemplary anti-cancer drugs that can be co-administered with the agents of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

According to a particular embodiment, the additional anti-cancer agents are rituximab, Bendamastin or a combination thereof.

As mentioned, the present inventors have shown that CXCR4 binding agents increase miR-15a and 16-1, which in turn leads to the down-regulation of Bcl-2. Thus, the present inventors propose that CXCR4 binding agents will be useful for treating subjects that are resistant to Bcl-2 inhibitors or antagonists.

Thus, according to another aspect of the present invention there is provided a method of rendering a BCl-2 antagonist resistant subject more susceptible to treatment with a BCl-2 antagonist comprising administering to the subject a therapeutically effective amount of a CXCR4 binding agent that increases the expression of miR-15a and/or 16-1, thereby treating the cancer.

BCl-2 antagonists and a CXCR4 binding agents have been described herein above.

A BCl-2 antagonist resistant subject is one that is less sensitive to the anticancer effects of BCl-2 antagonist than a non-resistant subject.

According to one embodiment, the BCl-2 antagonist resistant subject is insensitive to the anticancer effects of the BCl-2 antagonist.

According to one embodiment, the therapeutic effect of the BCl-2 antagonist is reduced by at least 10%, 20%, 30%, 40%, 50%, 60%, 70%, 80%, 90% or more in the BCl-2 antagonist resistant subject as compared to the effect in a subject that does not have resistance (e.g., a subject just starting BCl-2 antagonist therapy).

It will be appreciated that the BCl-2 antagonist resistant subject may be more resistant to the effects of a particular BCl-2 antagonist (e.g., ABT199) than other BCl-2 antagonists.

As mentioned herein above, the present inventors have shown that CXCR4 binding agents increase miR-15a and 16-1, thereby reducing the target genes—Bcl-2, cyclin D1 and Mcl-1. Reduction of such genes has been shown to be advantageous in treating a myriad of diseases including but not limited to cancer and neurodegenerative diseases.

Thus, according to still another aspect of the present invention there is provided a method of treating cancer or a neurodegenerative disease in a subject in need thereof, comprising administering to the subject a therapeutically effective amount of gp120 or CXCL12 (or peptides derived therefrom), thereby treating the cancer or neurodegenerative disease.

CXCL12 or gp120 polypeptides and peptides derived therefrom have been described herein above.

Examples of cancer that can be treated with gp120 or CXCL12 are described herein above.

According to a particular embodiment, the cancers are neuroblastoma, retinoblastoma bladder cancer, esophageal carcinoma, sarcomas, colorectal cancer, melanoma, parathyroid adenoma, and breast cancer.

According to another embodiment, the cancer is a solid tumor.

The term “neurodegenerative disease” is used herein to describe a disease which is caused by damage to the central nervous system. Exemplary neurodegenerative diseases which may be treated using the cells and methods according to the present invention include for example: Amyotrophic Lateral Sclerosis (ALS), Parkinson's disease, Multiple System Atrophy (MSA), Huntington's disease, Alzheimer's disease, Rett Syndrome, lysosomal storage diseases (“white matter disease” or glial/demyelination disease, as described, for example by Folkerth, J. Neuropath. Exp. Neuro., September 1999, 58:9), including Sanfilippo, Gaucher disease, Tay Sachs disease (beta hexosaminidase deficiency), other genetic diseases, multiple sclerosis (MS), brain injury or trauma caused by ischemia, accidents, environmental insult, etc., spinal cord damage, ataxia. In addition, the present invention may be used to reduce and/or eliminate the effects on the central nervous system of a stroke in a patient, which is otherwise caused by lack of blood flow or ischemia to a site in the brain of the patient or which has occurred from physical injury to the brain and/or spinal cord. Neurodegenerative diseases also include neurodevelopmental disorders including for example, autism and related neurological diseases such as schizophrenia, among numerous others.

The CXCL12 or gp120 polypeptides and peptides derived therefrom may be administered together with additional chemotherapeutic agents as further described herein above.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide or polypeptide agents described herein may be substituted, for example, by N-methylated amide bonds (—N(CH3)—CO—), ester bonds (—C(═O)—O—), ketomethylene bonds (—CO—CH2—), sulfinylmethylene bonds (—S(═O)—CH2—), α-aza bonds (—NH—N(R)—CO—), wherein R is any alkyl (e.g., methyl), amine bonds (—CH2—NH—), sulfide bonds (—CH2—S—), ethylene bonds (—CH2—CH2—), hydroxyethylene bonds (—CH(OH)—CH2—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides or polypeptides of some embodiments of the invention may also include one or more modified amino acids or one or more non-amino acid monomers (e.g., fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino | | | |
|---|---|---|---|
| acid | Code | Non-conventional amino acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl)carbamylmethyl- | Nnbhm |
| diphenylethyl)glycine | | glycine | |
| N-(3,3- | Nbhe | N-(N-(3,3-diphenylpropyl)carbamylmethyl- | Nnbhe |
| diphenylpropyl)glycine | | glycine | |
| 1-carboxy-1-(2,2-diphenylethylamino)cyclopropane | Nmbc | 1,2,3,4- | Tic |
| | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The present inventors contemplate using additional agents that are capable of binding CXCR4 and increasing the level of miRNA 15a or 16-1 for the treatment of cancer or neurodegenerative diseases. Such agents may be identified using screening assays as described herein below.

Candidate agents that may be screened include small molecule agents, polynucleotide agents, chemicals, antibiotic compounds known to modify gene expression, modified or unmodified polynucleotides (including oligonucleotides), polypeptides, peptides, small RNA molecules and miRNAs.

According to a particular embodiment the candidate agents are known to bind to CXCR4. Typically the candidate agents bind to CXCR4 with a Kd of less than one millimolar.

The agents of this aspect of the present invention are contacted with cell populations.

The cells may be isolated from any animal, including a mouse, a rat or a human. Alternatively, the cells may be part of a cell line.

Exemplary cells that can be used in the screening assays described herein express CXCR4 and/or miR15a/16-1 targets such as BCL-2, cyclin D1, MCL-1. MV4-11, RPMI-8226, PC3, K562, and others.

It will be appreciated that the methods of contacting according to this aspect of the present invention typically depend on the type of candidate agent being tested. Thus, for example a polynucleotide agent is typically contacted with cells together with a transfection agent. A small chemical is typically placed in the cell culture medium without additional agents.

To be considered a therapeutic agent, the candidate agents of the present invention typically binds to CXCR4 and up-regulates an activity or expression of miR-15a or 16-1, by at least 1.5 fold and more preferably by at least 2 fold. Methods of analyzing whether miR-15a or 16-1 is upregulated have been described herein above. Methods of analyzing the binding of the agent for CXCR4 may be affected using methods known in the art (e.g., by FRET, replacement assay with cold ligand).

Following selection of a candidate agent as a therapeutic agent for the treatment of cancer or a neurodegenerative disease, the agent may be tested—for example on an animal model for the disease and ultimately the agent may be tested in humans. Validation of therapeutic efficacy may then lead to the preparation of the candidate agent as a pharmaceutical composition.

According to yet another aspect of the present invention there is provided a method of treating HIV in a subject comprising administering to the subject a polynucleotide agent which is complementary to a nucleotide sequence of human miR-15a and/or 16-1, thereby treating the HIV in the subject.

Subjects that may be treated according to this aspect of the present invention have been infected with the HIV virus. The HIV may be HIV-1 or HIV-2. The subject may have an acute infection or a chronic HIV (asymptomatic HIV).

In some embodiments, the miR-15a or 16-1 inhibitor is an antagomir. An “antagomir” refers to a single stranded, double stranded, partially double stranded or hairpin structured oligomer or polymer of ribonucleic acid (RNA) or deoxyribonucleic acid (DNA) or both or modifications thereof, which is antisense with respect to its miRNA target.

Examples of antagomirs and other miRNA inhibitors are described in WO2009/20771, WO2008/91703, WO2008/046911, WO2008/074328, WO2007/90073, WO2007/27775, WO2007/27894, WO2007/21896, WO2006/93526, WO2006/112872, WO2007/112753, WO2007/112754, WO2005/23986, or WO2005/13901, all of which are hereby incorporated by reference. Custom designed Anti-miR™ molecules are commercially available from Applied Biosystems. Thus, in some embodiments, the antagomir is an Ambion® Anti-miR™ inhibitor. These molecules are chemically modified and optimized single-stranded nucleic acids designed to specifically inhibit naturally occurring mature miRNA molecules in cells.

Custom designed Dharmacon Meridian™ microRNA Hairpin Inhibitors are also commercially available from Thermo Scientific. These inhibitors include chemical modifications and secondary structure motifs. For example, Vermeulen et al. reports in U.S. Patent Publication 2006/0223777 the identification of secondary structural elements that enhance the potency of these molecules. Specifically, incorporation of highly structured, double-stranded flanking regions around the reverse complement core significantly increases inhibitor function and allows for multi-miRNA inhibition at subnanomolar concentrations. Other such improvements in antagomir design are contemplated for use in the disclosed methods.

In preferred embodiments, the disclosed antagomir includes a region of sufficient nucleotide length and sufficient complementarity to miR-15a or 16-1 that the antagomir forms a duplex with miR-15a or 16-1. Given the sequence of miR-15a and 16-1, an antagomir can be designed according to the rules of Watson and Crick base pairing.

Thus, the antagomir can be an antisense oligonucleotide having a single-stranded nucleic acid sequence that is complementary to at least 8, 9, 10, 11, 12, 13, 14, 15, 16, 17, 18, 19, 20, 21, 22, 23, or 24 contiguous nucleotides in miR-15a or 16-1, wherein the antisense oligonucleotide forms a duplex with miR-15a or 16-1 under physiological (or stringent) conditions.

The antagomir can include an antisense oligonucleotide having a length of at least 8 contiguous nucleotides. Therefore, the antisense oligonucleotide can have 8, 9, 10, 11, 12, 13, 14, 15, 16, 17, 18, 19, 20, 21, 22, 23, or 24 contiguous nucleotides. The oligonucleotide is preferably less than 30 contiguous nucleotides in length. The oligonucleotide can be less than 25, 26, 27, 28, 29, 30, 31, 32, 33, 34, 35, 36, 37, 38, 39, 40 contiguous nucleotides in length.

The disclosed antagomirs can include an antisense oligonucleotide having a region that is at least partially, and in some embodiments fully, complementary to miR-15a or 16-1. It is not necessary that there be perfect complementarity between the antagomir and the target, but the correspondence must be sufficient to enable the antisense oligonucleotide to duplex with miR-15a or 16-1 and subsequently reduce its activity. The disclosed antagomir can include an antisense oligonucleotide having a region that is at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to miR-15a or 16-1.

In some embodiments, there will be nucleotide mismatches in the region of complementarity. In a preferred embodiment, the region of complementarity will have no more than 1, 2, 3, 4, or 5 mismatches. In some embodiments, the antagomir is “exactly complementary” to miR-15a or 16-1. Thus, in one embodiment, the antagomir can anneal to miR-33 to form a hybrid made exclusively of Watson-Crick base pairs in the region of exact complementarity. Thus, in some embodiments, the antagomir specifically discriminates a single-nucleotide difference. In this case, the antagomir only inhibits miR-15a or 16-1 activity if exact complementarity is found in the region of the single-nucleotide difference.

The disclosed antagomirs can be modified as described herein above in the section which recites modifications for nucleic acid agents. Additional modifications contemplated by the present inventors are provided in US Patent application No. 20140080899, the contents of which are incorporated herein by reference.

An exemplary inhibitor of miR-15a is the miRIDIAN microRNA hsa-miR-15a-5p hairpin inhibitor (Dharmacon) and an exemplary inhibitor of miR-16-1 is the miRIDIAN microRNA hsa-miR-16-5p hairpin inhibitor (Dharmacon).

It is expected that during the life of a patent maturing from this application many relevant Bcl2 antagonists and CXCR4 binding agents will be developed and the scope of the terms “Bcl2 antagonists” and “CXCR4 binding agents” are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Overexpression of CXCR4 Leads to Up Regulation of mir15a and mir16-1

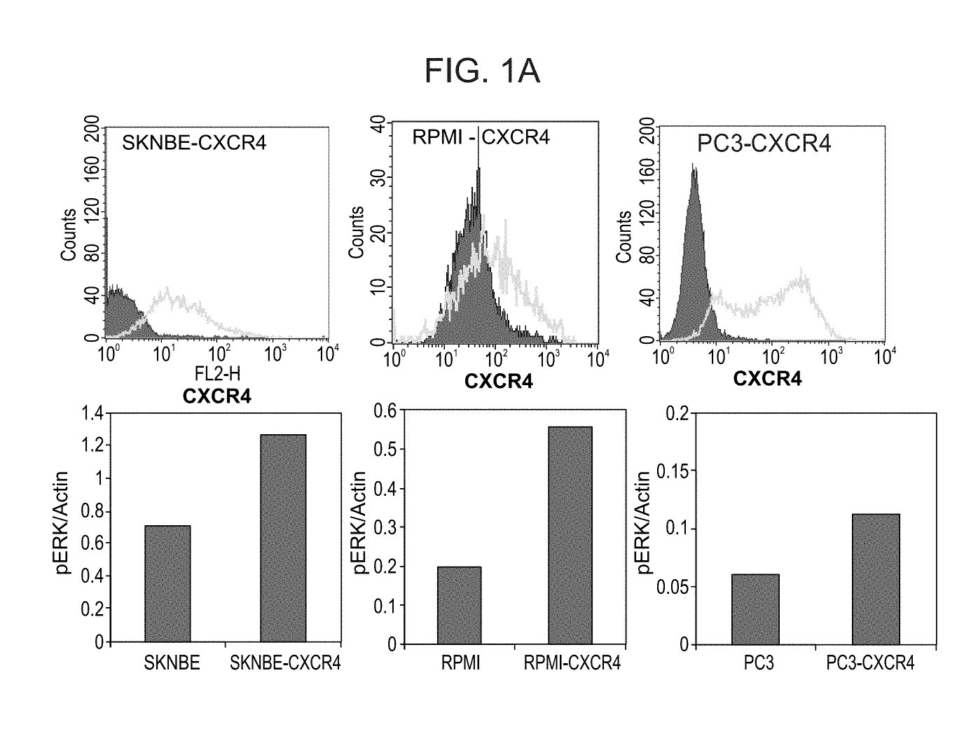

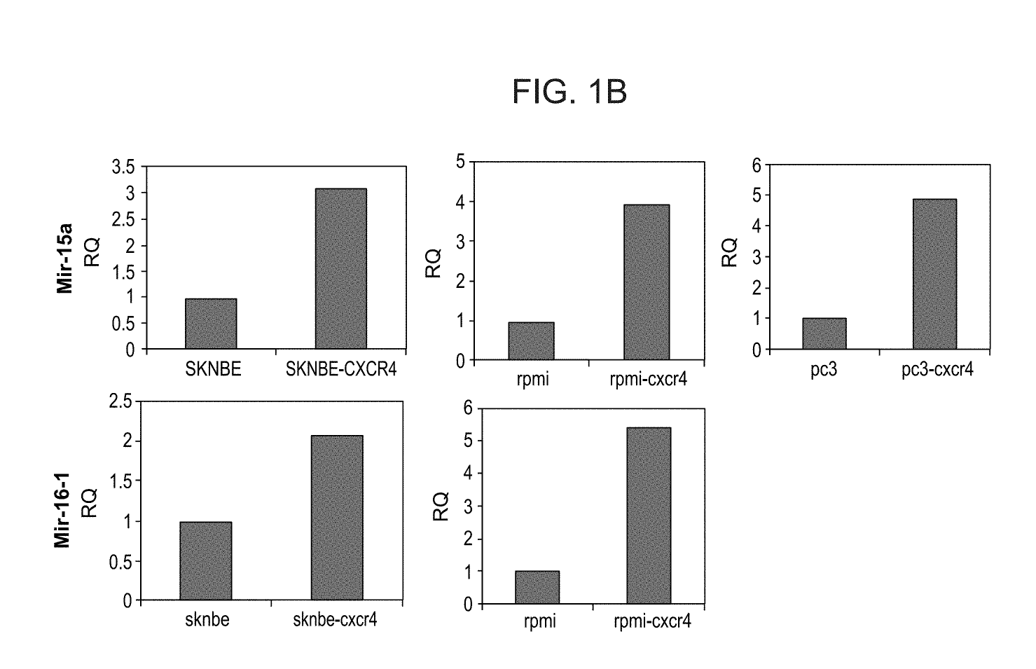

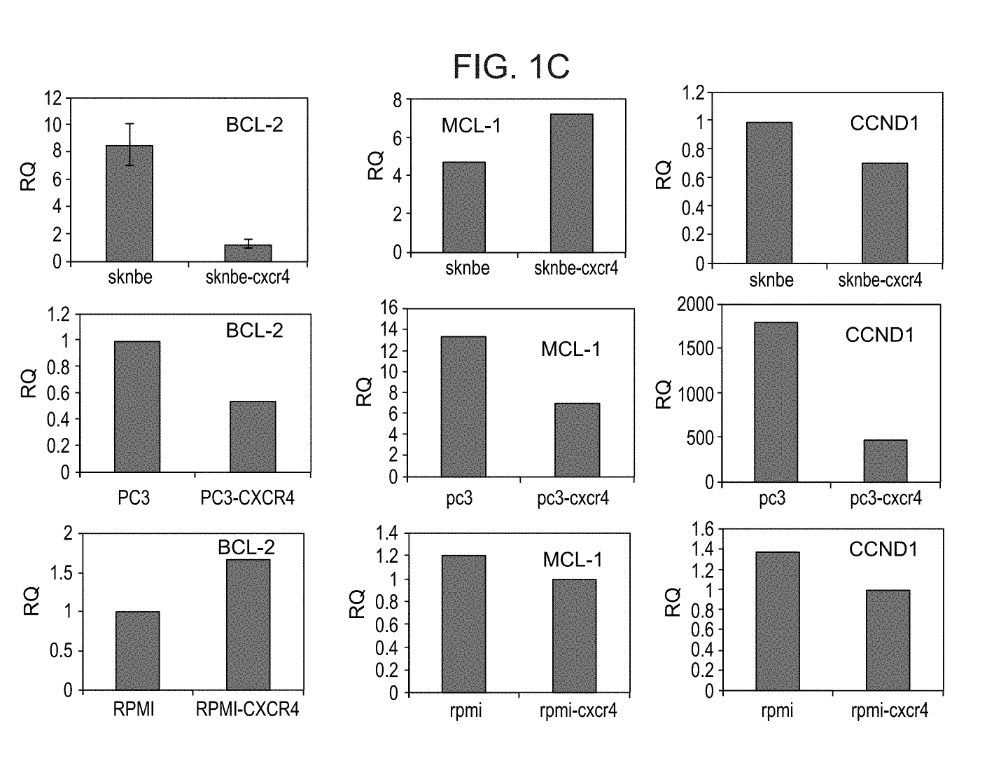

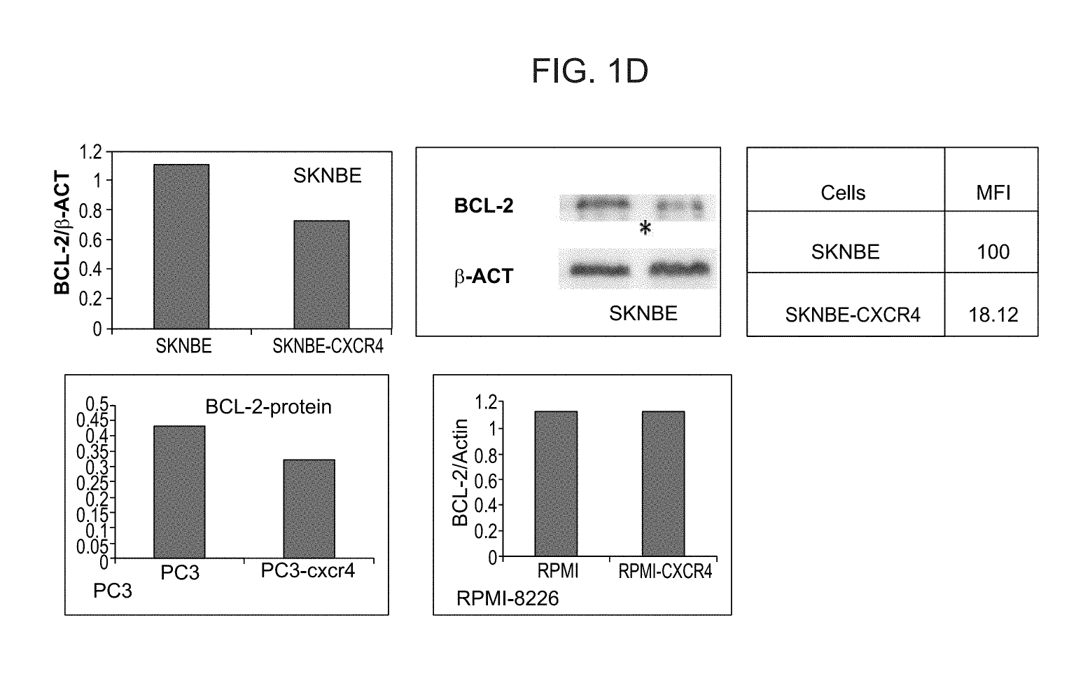

CXCR4 is overexpressed in the majority of tumors and its expression often correlates with bad prognosis. However, in the majority of tumor cell lines grown in culture, CXCR4 is expressed on the cell surface at low levels. It was found that overexpression of CXCR4 in various tumor cells increases their tumorigenicity in vitro and in vivo by increasing their survival signals through the ERK (FIG. 1A) signaling pathways. This finding is supported by numerous publications reporting that CXCL12/CXCR4 axis and its downstream signals PI3K/AKT PErk1/2 pathways play an important role in tumor progression and metastasis.

Unexpectedly, it was now found that over expressing CXCR4 in tumor cells leads to up regulation of mir15 and 16 (FIG. 1B). MicroRNAs encoded by the mir-15a/16-1 locus (mir-15a and mir-16-1-1) function as tumor suppressors. Expression of these miRNAs are downregulated in CLL, melanoma, colorectal cancer, bladder cancer and other solid tumors. mir-15a/16 cluster targets multiple oncogenes, including BCL2, Cyclin D1, MCL1 and others. Indeed, up regulation of CXCR4 and mir15/16 lead to down regulation of mir15/16 target oncogenes mRNA; BCL-2, MCL-1, and cyclin D1 in a cell specific manner (FIG. 1C).

In the SKNBE neuroblastoma cells, overexpression of CXCR4 strongly upregulated mir15/16 and significantly downregulated BCL2 mRNA expression levels. Downregulation of BCL2 protein levels was also evident by Western Blot as well by internal FACS analysis (FIG. 1D). In correlation with the mRNA levels, reduced levels of BCL2 were observed in the PC3 cells but not RPMI-8226 cells (FIG. 1D).

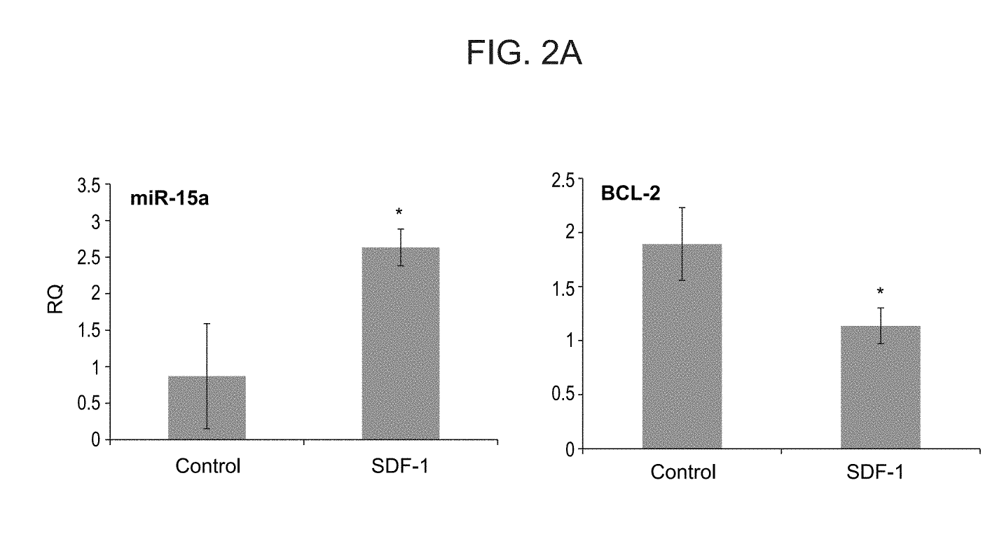

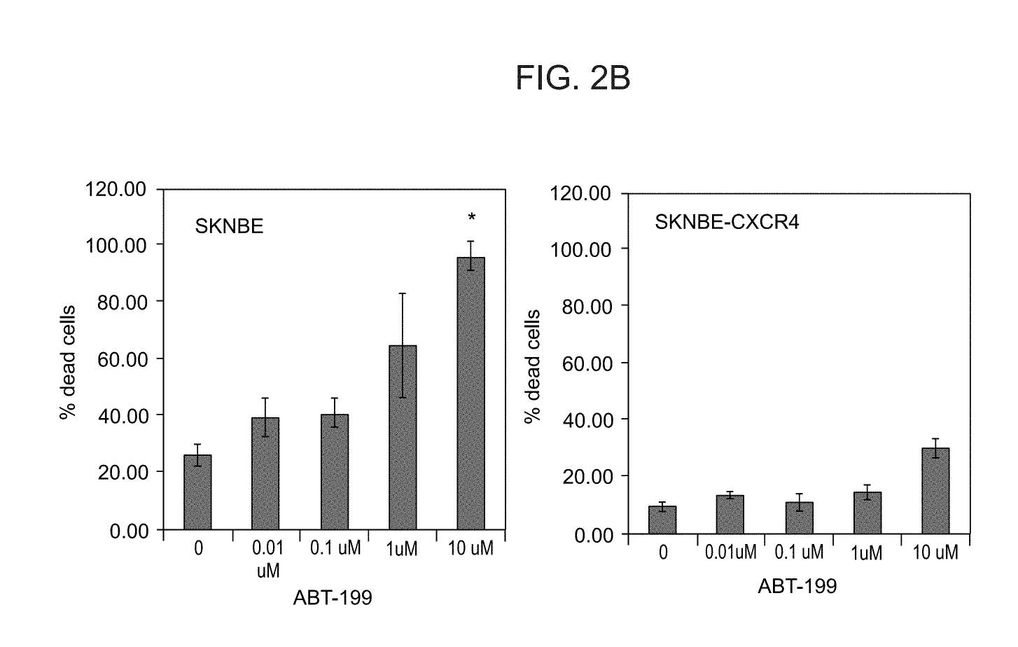

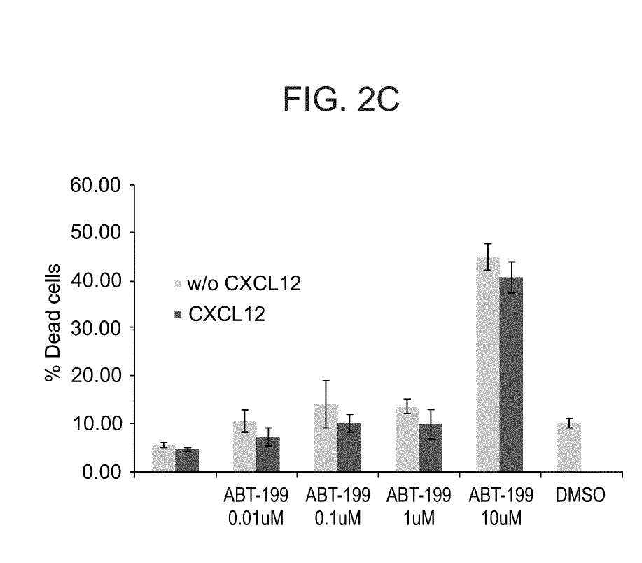

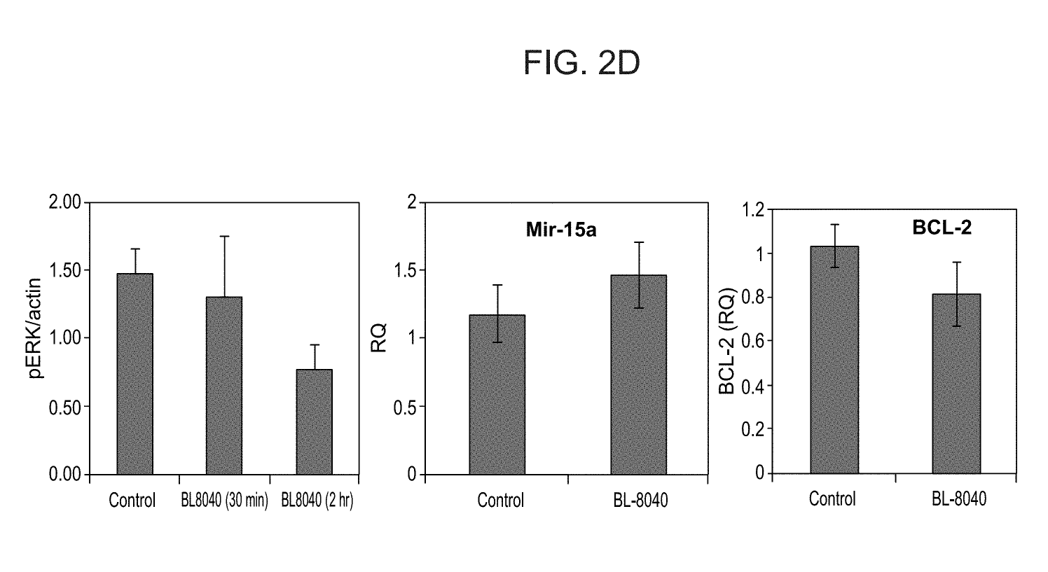

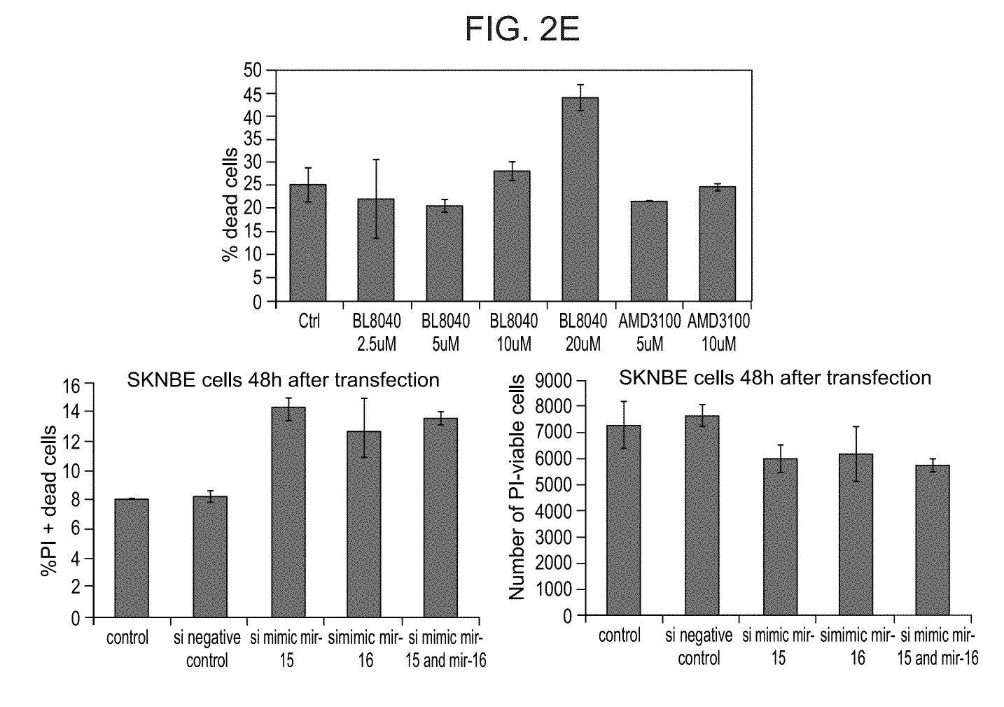

To study the cross talk between the CXCL12/CXCR4 axis and mir15/BCL2 axis the present inventors tested the effect of CXCL12 on mir15/BCL2 expression in SKNBE neuroblastoma cells. It was found that CXCL12 induces the expression of mir15 and downregulates the expression of BCL2 in SKNBE neuroblastoma cells (FIG. 2A). It was found that over-expression of CXCR4 or stimulating SKNBE neuroblastoma cells with the CXCR4 ligand upregulates mir15/16 and down regulates BCL2. Next, the present inventors examined whether down regulation of BCL2 that follows the up regulation of CXCR4 desensitizes the cells to the specific BCL2 inhibitor, ABT199. Indeed, it was found that overexpression of CXCR4 transformed the cells to be un-resistant to ABT199 (FIG. 2B). In addition, treatment of SKNBE cells with CXCL12 attenuated the apoptosis induced by treatment the cells with ABT199 (FIG. 2B) while pre-treatment of SKNBE cells with CXCL12 attenuated the apoptosis induced by treatment with ABT199 (FIG. 2C) Interestingly, treatment of SKNBE with the CXCR4 binding agent BL8040 inhibited ERK signaling as well as induced mir-15a expression and reduced BCL-2 expression levels in these cells (FIG. 2D). The inhibition of these survival signals by BL8040 induces SKNBE cell death. Indeed up-regulation of mir-15a by transfection of cells with mir-15a or mir-16-1-1 induced SKNBE cell death (FIG. 2E).

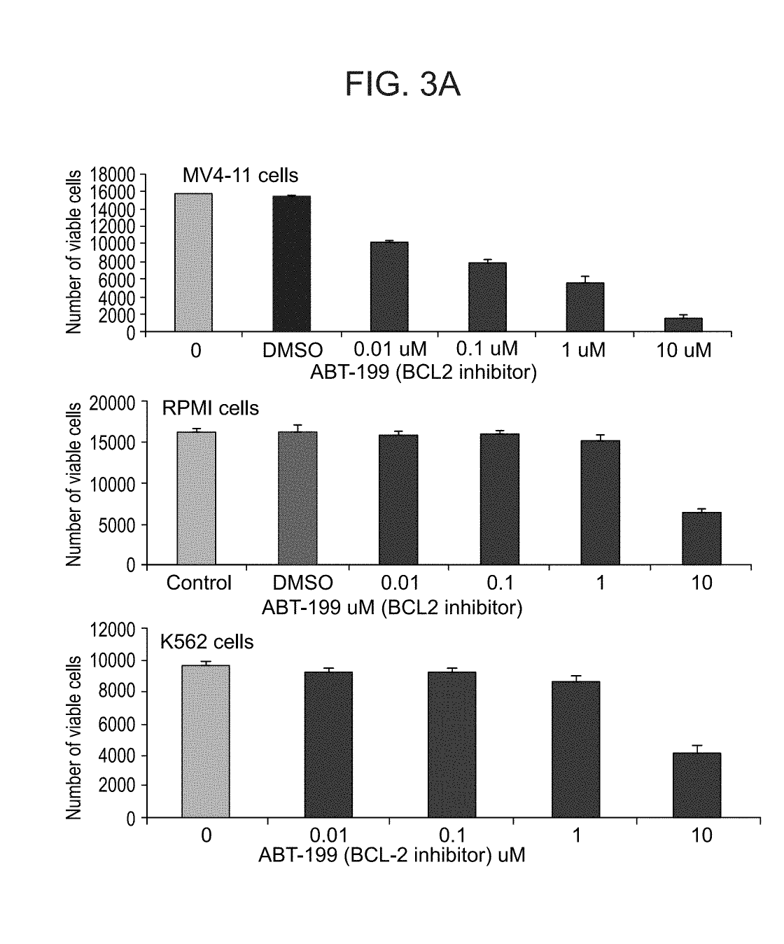

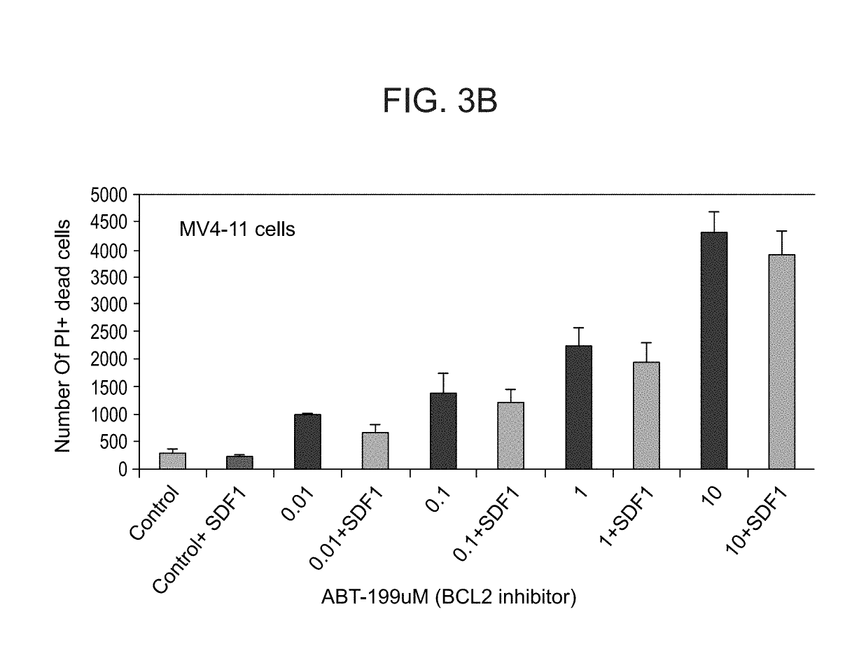

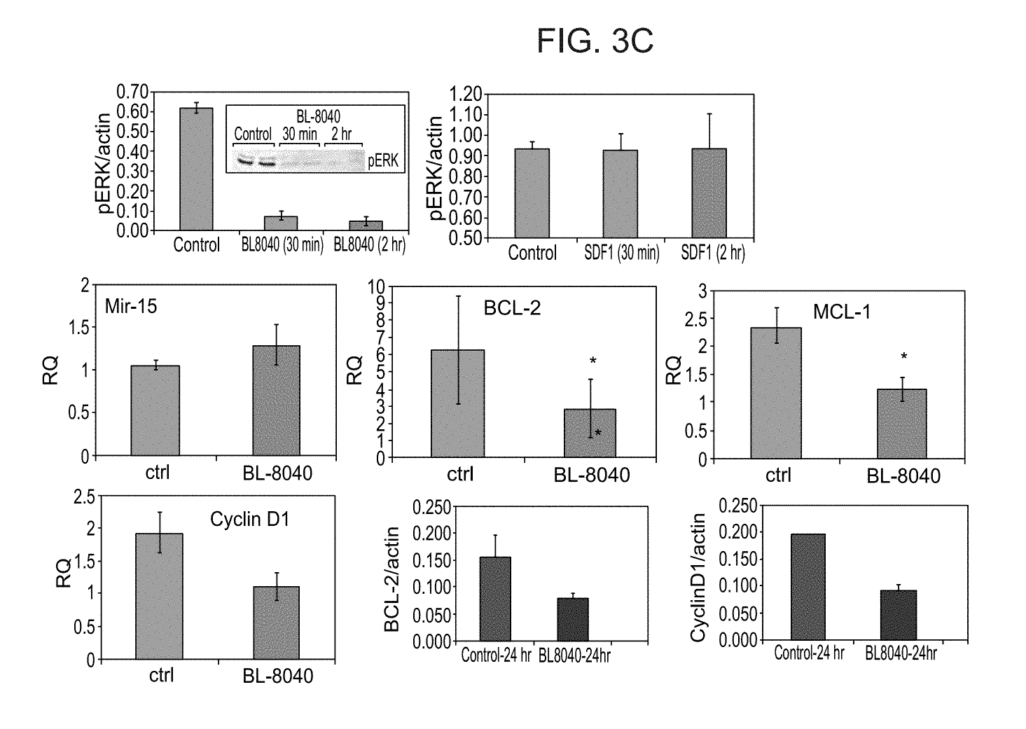

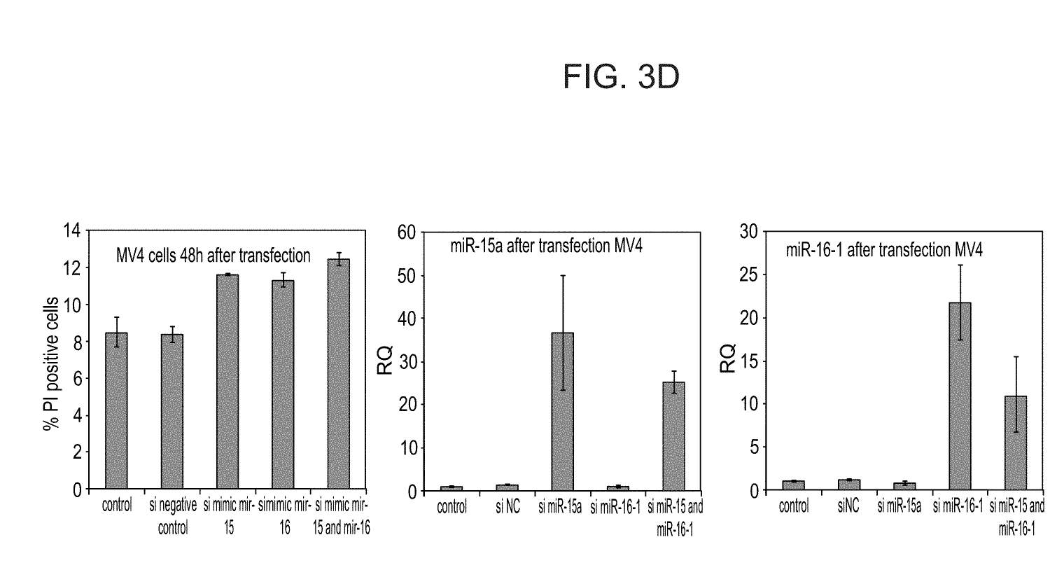

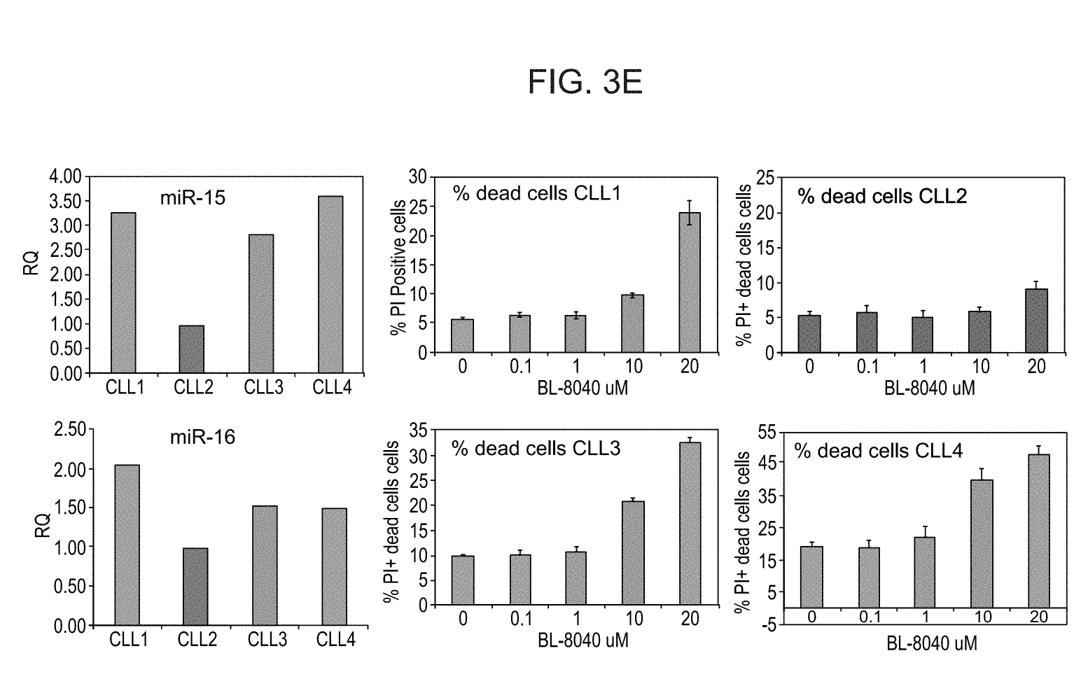

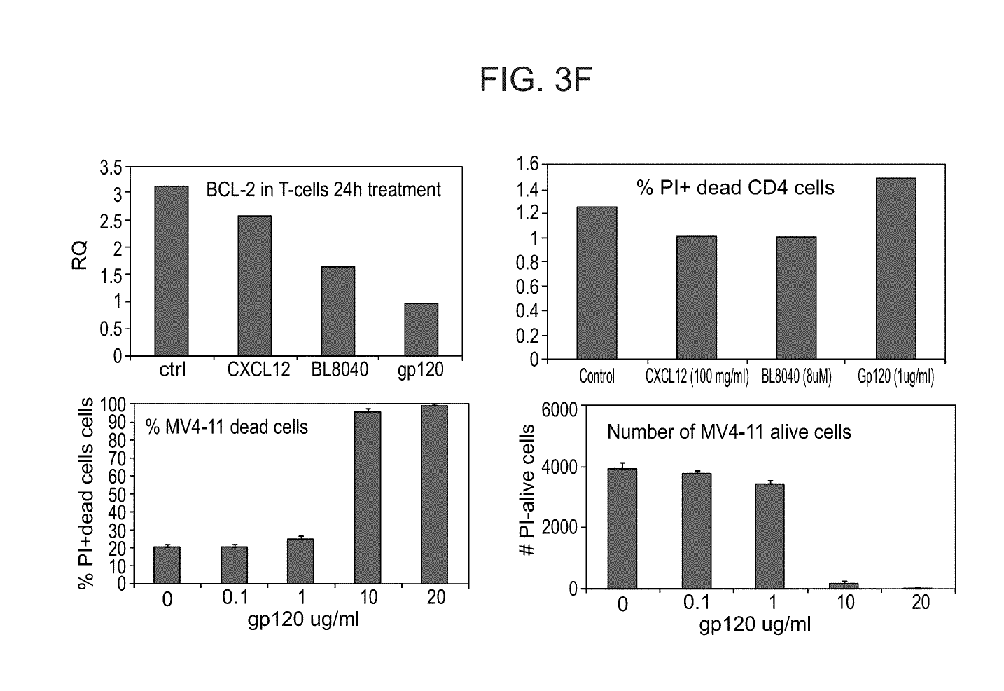

To further study the cross talk between the CXCL12/CXCR4 axis and mir-15a/BCL2 axis, the AML cell line MV4-11 was analyzed. It was found that these cells are dependent on BCL-2 for their survival in vitro and can be induced to apoptosis using the ABT-199 at IC50 of 10 nM (FIG. 3A). Similar to the results obtained with SKNBE cells, treatment of MV4-11 cells with CXCL12 also attenuated the apoptosis induced by treatment with ABT199 (FIG. 3B). The CXCR4 binding agent BL8040 induced rapid and dose dependent apoptosis of MV4-11 cells inhibited ERK signaling as well as induced mir-15a expression and reduced significantly BCL2/MCL-1/and cyclinD1 expression levels (FIG. 3C). Furthermore, transfection of these cells with mir-15a or mir-16-1-1 also induces their cell death (FIG. 3D).

These results suggest that the CXCR4 binding agent BL8040 inhibit ERK signaling through the CXCR4 receptor, up regulate mir-15a/16-1 which in turn further deprived the cells from survival signals by down regulating the oncogenes BCL2/MCL1/and cyclin D1.

B-cell chronic lymphocytic leukemia (CLL) is the most common adult leukemia.

The most common chromosomal abnormalities detectable by cytogenetics include deletion at 13q. In 2002, it was discovered that a microRNA cluster mir-15a/mir-16-1-1 (mir-15a/16) is the target of 13q deletions in CLL. mir-15a/16 cluster targets multiple oncogenes, including BCL2, Cyclin D1, MCL1 and others. The most important target of mir-15a/16 in CLL is arguably BCL2, as BCL2 is overexpressed in almost all CLLs. It was found that in CLL cells that are deleted from mir-15a/16 (CLL patient 1), BL8040 was unable to induce cell death (FIG. 3D). This suggests a critical role for this pathway in the induction of tumor cell death by this anti CXCR4 antagonist BL8040 (FIG. 3E).

In AIDS patients, the absolute number of CD8+ and CD4+ T lymphocytes is slowly decreased in peripheral blood and their turnover rate is increased, suggesting that there is more cell renewal and cell death occurring. However the exact mechanisms that regulate this process are unknown. In addition, the human immunodeficiency virus type 1 (HIV-1) glycoprotein gp120 causes neuronal cell death; however, the molecular mechanisms of the neurotoxic effect remain largely unresolved. It was found that gp120 up regulates mir15/16 which in turn further deprived the cells from survival signals by down regulating BCL2 in CD4+ cells (FIG. 3E). Furthermore, gp120, but not CXCL12 or BL8040, induced small but significant apoptosis of these cells (FIG. 3F). Surprisingly, the effect of gp120 on the survival of MV4-11 AML cells was dramatic (FIG. 3F).

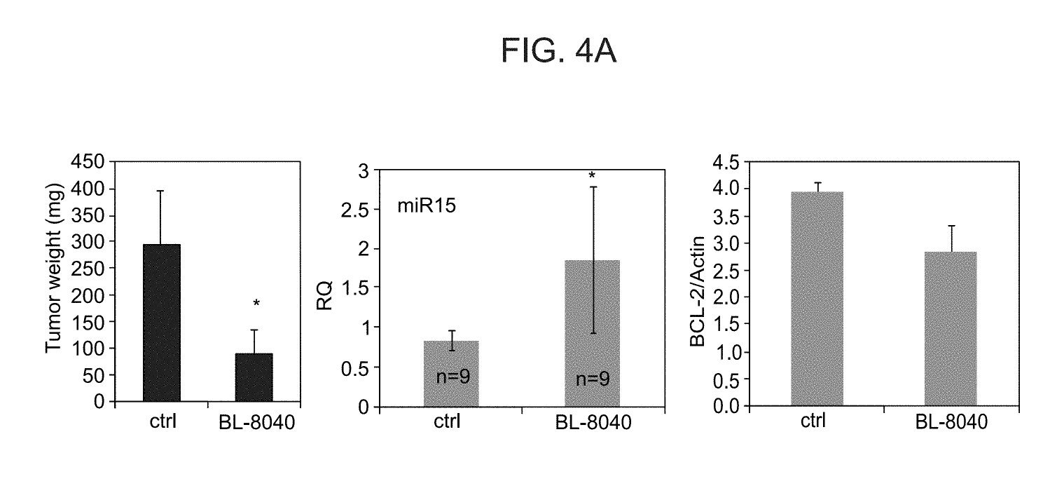

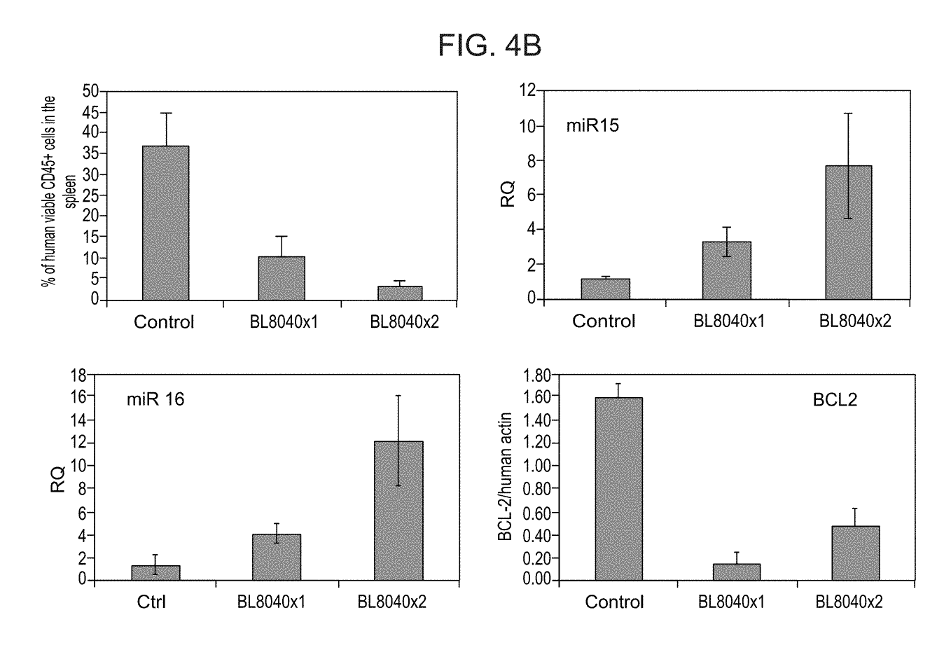

To further evaluate the effect of BL8040 on tumor growth mir-15a/16-1 and BCL2 in vivo expression, neuroblastoma and leukemia mouse models in NSG mice were established using the SKNBE and MV4-11 cells. In the vivo orthotopic neuroblastoma (NB) model in NSG mice 0.5×10⁶ SKNBE cells were injected into the mice adrenal. One week later BL-8040 was injected daily for 3 weeks at 400 ug/mouse treatment with BL-8040 induced tumor cell death and significantly inhibits tumor growth (FIG. 4A). In association, BL8040 treatment significantly increase mir15 and down regulated BCL2 in vivo (FIG. 4A).

In the in-vivo AML model with MV4-11 cells, 1×10⁶ cells were IV injected into NSG mice. Three week later BL-8040 was SC injected daily once (BL-8040×1) or twice (BL-8040×2) at 400 ug/mouse. 24 hr after the last injection mice were sacrificed. BL8040 was found to induce AML apoptosis and reduce dramatically the number of AML cells in the spleen as demonstrated by FACS analysis following staining with human CD45 fluorescence antibody. Reduction in AML cells in the spleen was associated with a significant increase in miR15/16 and down regulation of BCL2 in human cells (FIG. 4B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating cancer in a subject in need thereof, the method comprising administering to the subject:
(i) a therapeutically effective amount of an inhibitory agent that down-regulates an amount of a polypeptide selected from the group consisting of BCL-2, MCL-1 and cyclin D1; and
(ii) a therapeutically effective amount of a CXCR4 binding agent that increases the expression of miR-15a and/or 16-1, thereby treating cancer.

2-7. (canceled)

8. The method of claim 1, wherein said inhibitory agent is a BCl-2 antagonist.

9. The method of claim 8, wherein said BCl-2 antagonist is selected from the group consisting of Oblimersen, SPC-2996, RTA-402, Gossypol, AT-101, Obatoclax mesylate, A-371191, A-385358, A-438744, ABT-737, ABT-199, AT-101, BL-11, BL-193, GX-15-003, 2-Methoxyantimycin A3, HA-14-1, KF-67544, Purpurogallin, TP-TW-37, YC-137 and Z-24.

10. The method of claim 9, wherein said BCl-2 antagonist is ABT-199.

11. The method of claim 1, further comprising an additional chemotherapeutic agent.

12. The method of claim 11, wherein said additional chemotherapeutic agent is rituximab and Bendamastin.

13. The method of claim 1, wherein said CXCR4 binding agent binds to the CXCL12 binding site of CXCR4.

14. The method of claim 1, wherein said agent is BL8040.

15. The method of claim 1, wherein said CXCR4 binding agent is gp120 or CXCL-12.

16-17. (canceled)

18. A method of rendering a BCl-2 antagonist resistant subject more susceptible to treatment with a BCl-2 antagonist, the method comprising administering to the subject: a CXCR4 binding agent that increases the expression of miR-15a and/or 16 in the subject.

19. The method of claim 18, wherein said BCl-2 antagonist is selected from the group consisting of Oblimersen, SPC-2996, RTA-402, Gossypol, AT-101, Obatoclax mesylate, A-371191, A-385358, A-438744, ABT-737, ABT-199, AT-101, BL-11, BL-193, GX-15-003, 2-Methoxyantimycin A3, HA-14-1, KF-67544, Purpurogallin, TP-TW-37, YC-137 and Z-24.

20. The method of claim 18, wherein said BCl-2 antagonist is ABT-199.

21. The method of claim 18, wherein said CXCR4 binding agent binds to the CXCL12 binding site of CXCR4.

22. The method of claim 18, wherein said CXCR4 binding agent is BL8040.

23. The method of claim 18, wherein said CXCR4 binding agent is gp120 or CXCL-12.

24. The method of claim 18, wherein the subject has a cancer selected from the group consisting of a hematological cancer, neuroblastoma, retinoblastoma bladder cancer, esophageal carcinoma, sarcomas, colorectal cancer, melanoma, parathyroid adenoma and breast cancer.

25. The method of claim 24, wherein said hematological cancer is selected from the group consisting of of Chronic Myelogenous Leukemia (CML), CML accelerated phase, or blast crisis, multiple myeloma, Hypereosinophilic Syndrome (HES), myelodysplastic syndrome (MDS), acute lymphocytic leukemia (ALL), acute myeloid leukemia (AML), acute promyelocytic leukemia (APL), chronic lymphocytic leukemia (CLL), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myeloproliferative disorders (MPD), multiple myeloma, (MM) and myeloid sarcoma.

26. A method of treating cancer or a neurodegenerative disease in a subject in need thereof, the method comprising administering to the subject a therapeutically effective amount of gp120 or CXCL12, thereby treating cancer or neurodegenerative disease, with the proviso that the cancer is not prostate cancer.

27. The method of claim 26, wherein the cancer is a hematological cancer.

28. The method of claim 26, wherein the neurodegenerative disease is selected from the group consisting of Alzheimer's disease (AD), Parkinson's disease, and frontotemporal dementias (FTD).

29-33. (canceled)

34. A method of treating HIV in a subject the method comprising administering to the subject a polynucleotide agent which is complementary to a nucleotide sequence of human miR-15a and/or 16-1, thereby treating HIV in a subject.


SEQUENCE LISTING (14 sequences)

SEQ ID NO: 1 - RNA, 9 nt, Artificial Sequence
  oligonucleotide sequences that can be used to form a loop
uucaagaga

SEQ ID NO: 2 - RNA, 9 nt, Artificial Sequence
  oligonucleotide sequences that can be used to form a loop
uuuguguag

SEQ ID NO: 3 - RNA, 22 nt, Artificial Sequence
  miRNA
uagcagcacauaaugguuugug

SEQ ID NO: 4 - RNA, 22 nt, Artificial Sequence
  miRNA
uagcagcacguaaauauuggcg

SEQ ID NO: 5 - DNA, 18 bp, Artificial Sequence
  18-mer antisense  oligodeoxynucleotide
  misc_feature (1)..(18): phosphorothioate oligodeoxynucleotides backbone
tctcccagcgtgcgccat

SEQ ID NO: 6 - DNA, 16 bp, Artificial Sequence
  16-mer antisense oligonucleotide
  misc_feature (1)..(16): phosphorothioate oligodeoxynucleotide backbone
  misc_feature (1)..(2): locked nucleic acid (LNA) nucleotides
  misc_feature (14)..(15): locked nucleic acid (LNA) nucleotides
ctcccaacgtgcgcca

SEQ ID NO: 7 - DNA, 21 bp, Artificial Sequence
  siRNA
gcatgcggcctctgtttgatt

SEQ ID NO: 8 - DNA, 19 bp, Artificial Sequence
  siRNA
tccaaggcatgcttcggaa

SEQ ID NO: 9 - DNA, 21 bp, Artificial Sequence
  siRNA
ccagagugaucaagugugatt

SEQ ID NO: 10 - DNA, 22 bp, Artificial Sequence
  oligonucleotide probe
uagcagcacauaaugguuugug

SEQ ID NO: 11 - DNA, 22 bp, Artificial Sequence
  oligonucleotide probe
uagcagcacguaaauauuggcg

SEQ ID NO: 12 - protein, 89 aa, Homo sapiens
  misc_feature: Human SDF-1 alpha
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 13 - protein, 89 aa, Mus musculus
  misc_feature: Murine SDF-1 alpha
MDAKVVAVLALVLAALCISDGKPVSLSYRCPCRFFESHIARANVKHLKILNTPNCALQIVARLKNNNRQVCIDPKLKWIQEYLEKALNK

SEQ ID NO: 14 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
XRXCYXKXPYRXCR